SEQUENCE LISTING (1 sequence)

SEQ ID NO: 1 - protein, 14 aa, Artificial Sequence
  synthetic construct; MAPKK2 derived peptide
GYARRRRRRRRVLR

(19) United States
(12) Patent Application Publication
(10) Pub. No.: US 2015/0087605 A1
(43) Pub. Date: Mar. 26, 2015

(54) Compositions and Methods Comprising Carboxylic Acid-Containing Small Molecules

(71) Applicant: University of Central Florida Research Foundation, Inc., Orlando, FL (US)

(72) Inventors: J. Manuel Perez Figueroa, Orlando, FL (US); Oscar Santiesteban, Orlando, FL (US); Charalambos Kaittanis, Orlando, FL (US)

(21) Appl. No.: 14/491,374

(22) Filed: Sep. 19, 2014

Related U.S. Application Data
(60) Provisional application No. 61/880,706, filed on Sep. 20, 2013.

(51) Int. Cl. A61K 31/192; A61K 31/405; A61K 31/195; A61K 31/196; A61K 31/495; G01N 33/573; A61K 31/407; A61K 31/341; A61K 31/4375; A61K 38/06; G01N 33/58; A61K 45/06; A61K 31/704

(52) U.S. Cl. CPC A61K 31/192; A61K 45/06; A61K 31/405; A61K 31/195; A61K 31/196; A61K 31/495; A61K 31/704; A61K 31/407; A61K 31/341; A61K 31/4375; A61K 38/06; G01N 33/587; G01N 33/573; G01N 2333/954
USPC 514/21.9; 514/569; 514/419; 514/563; 514/562; 514/255.04; 514/34; 514/411; 514/471; 514/300; 436/501; 435/7.4; 506/9

(57) ABSTRACT

Disclosed are compositions and methods for treating anthrax, inhibiting anthrax toxins and inhibiting anthrax toxin-induced cytotoxicity. Carboxylic acid-containing small molecules can be used in the methods and compositions disclosed herein, for example, sulindac and derivatives thereof may be used. Methods of screening for carboxylic acid-containing small molecules that can be used to treat anthrax are disclosed. Targeting the anthrax toxin reduces the risks of anthrax spores.

### CROSS-REFERENCE TO RELATED APPLICATIONS

This application claims benefit of U.S. Provisional Application No. 61/880,706, filed Sep. 20, 2013. Application No. 61/880,706, filed Sep. 20, 2013, is hereby incorporated herein by reference in its entirety.

### STATEMENT REGARDING FEDERALLY SPONSORED RESEARCH

This invention was made with government support under GM084331 awarded by the National Institutes of Health. The government has certain rights in the invention.

### REFERENCE TO SEQUENCE LISTING

The Sequence Listing submitted Sep. 19, 2014 as a text file named “26150_{—}0038U2_Sequence_Listing,” created on Aug. 11, 2014, and having a size of 597 bytes is hereby incorporated by reference pursuant to 37 C.F.R. §1.52(e)(5).

### FIELD OF THE INVENTION

The disclosure generally relates to compositions and methods for treating anthrax, inhibiting anthrax toxin activity, and screening for effective compounds.

### BACKGROUND

Anthrax is a disease caused by the bacterium Bacillus anthracis and is extremely lethal to both humans and animals. Anthrax is considered one of the greatest biological warfare threats.

Anthrax can occur via cutaneous or inhalation infection, the inhalation cases being the most deadly. Current treatments for inhalation anthrax are limited. Antibiotics have proven very efficient in eliminating the bacterial infection, but they lack the ability to destroy or inhibit the toxins released by the bacteria. This is a significant problem, as the lethal factor (LF) toxin can remain active in the body for days after the infection has been eliminated causing further macrophage death. Therefore, inhibitors of the LF toxin can be used in addition to antibiotics for a more effective treatment of Anthrax infection. Over the last decade, several inhibitors of the enzymatic and pathogenic activity of LF have been identified. In order to identify inhibitors of LF a variety of approaches have been utilized, such as library screenings, Mass Spectroscopy-based mining and scaffold-based NMR searches. Results from these screenings have yielded a variety of novel small molecules that inhibit LF at low micromolar concentrations. Although valuable, these small molecules are of low clinical translation with regards to treating LF, as pharmaceutical companies have a low incentive to spend time and invest millions of dollars to further develop, test and apply for FDA approval of these drug candidates, due to the low incidence of inhalation anthrax in the general population. What is needed are compositions and treatments for anthrax.

### BRIEF SUMMARY

Disclosed are compositions and methods for inhibiting the anthrax LF toxin. Also disclosed are screening methods for identifying LF inhibitors.

Disclosed are methods of inhibiting anthrax lethal factor (LF) toxin activity comprising administering an effective amount of one or more carboxylic acid-containing small molecules to a subject in need thereof. Disclosed are compositions comprising one or more carboxylic acid-containing small molecules.

Disclosed are methods of treating a subject having anthrax comprising administering a therapeutically effective amount of a carboxylic acid-containing small molecule to the subject, wherein the effective amount of the carboxylic acid-containing small molecule reduces or inhibits lethal factor protease activity.

Also disclosed are methods of decreasing or inhibiting anthrax toxin-induced cytotoxicity comprising administering an effective amount of a carboxylic acid-containing small molecule to a subject in need thereof.

Carboxylic acid-containing small molecules used in disclosed methods and compositions may be FDA-approved for a condition other than treating anthrax. A carboxylic acid-containing small molecule of the present invention may comprise, but is not limited to Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicinderivatives thereof, or mixtures thereof. In disclosed methods and compositions, the carboxylic acid-containing small molecule can be Sulindac, fusaric acid, a derivative thereof or mixtures thereof. A sulindac derivative can be a metabolic derivative and the metabolic derivate can be sulindac sulfide or sulindac sulfone.

Disclosed methods of inhibiting anthrax lethal factor (LF) toxin activity comprising administering an effective amount of one or more carboxylic acid-containing small molecule to a subject in need thereof and may comprise the carboxylic acid-containing small molecule binding to LF present in the subject. In particular, binding can occur at the active site of LF.

Disclosed methods of treating a subject having anthrax comprising administering a therapeutically effective amount of one or more carboxylic acid-containing small molecules to the subject, wherein the effective amount of the one or more carboxylic acid-containing small molecules reduces or inhibits lethal factor protease activity can comprise administering one or more antibacterial compounds or other therapeutic agents, wherein an antibacterial compound targets and is effective in killing or inhibiting the bacteria that causes anthrax.

Disclosed methods of decreasing or inhibiting anthrax toxin-induced cytotoxicity comprising administering an effective amount of a carboxylic acid-containing small molecule to a subject in need thereof can occur when the cytotoxicity occurs in macrophages.

Disclosed methods may be performed when the subject in need thereof is a subject infected with anthrax. For example, the anthrax can be inhalation anthrax.

Also disclosed are methods of screening comprising a) conjugating a magnetic relaxing (MR) nanosensor to a ligand to form a ligand-MR nanosensor complex; b) contacting the ligand-MR nanosensor complex with a sample containing possible ligand targets; and c) determining the magnetic resonance in the sample, wherein a change in magnetic resonance is indicative of a target bound to the ligand-MR nanosensor complex. In some instances, the change can be an increase in magnetic resonance. In some instances the change can be a decrease in magnetic resonance.

In disclosed screening methods, the ligand can be a known compound. In one aspect, the ligand can be one or more carboxylic acid-containing molecules, such as sulindac. In one aspect, the ligand can be an FDA-approved drug, compound or biological molecule.

Screening methods involve an MR nanosensor conjugated to a ligand, wherein the MR nanosensor comprises an iron oxide nanoparticle. The iron oxide nanoparticle can be coated with polyacrylic acid. Disclosed are compositions comprising ligand-MR nanosensors. Disclosed are compositions comprising carboxylic acid-containing small molecules-MR nanosensors.

Disclosed are screening methods comprising a) conjugating an MR nanosensor to a ligand to form a ligand-MR nanosensor complex; b) contacting the ligand-MR nanosensor complex with a sample containing possible ligand targets; and c) determining the magnetic resonance in the sample, wherein a change in magnetic resonance is indicative of a target bound to the ligand-MR nanosensor complex, wherein the target can be an anthrax toxin. In particular, the anthrax toxin can be lethal factor. In some instances, the change can be an increase in magnetic resonance. In some instances the change can be a decrease in magnetic resonance.

Additional advantages of disclosed methods and compositions will be set forth in part in the description which follows, and in part will be understood from the description, or may be learned by practice of disclosed method and compositions. The advantages of disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of disclosed methods and compositions and together with the description, serve to explain the principles of disclosed methods and compositions.

FIGS. 1A and B show representative spectral characteristics of the small molecules before and after attachment to the nanoparticle. The successful attachment of all the small molecules to the surface of the nanoparticle was verified by either fluorescence or absorbance emission depending on the spectral characteristic of the nanoparticle that was attached. a: Fluorescence spectra of naproxen and its corresponding bMR at 330 nm. B: Absorbance spectra of sulindac and it corresponding bMRs.

FIGS. 2A-C exemplify the screening of the small-molecule library. (a) In the first part of the screening assay, the corresponding bMR nanosensors are incubated with increasing concentrations of the toxin and the changes in T2 (ΔT2ₜₒₓᵢₙ) are recorded. As the concentration of the toxin increases so does the ΔT2. In the second part of our assay, the bMR nanosensors that successfully bind to the toxin are then incubated with in increasing concentrations of the free small molecule as competitor in order to assess the KD of that particular interaction. (b) Graphical representation and formula used to calculate the changes in magnetic relaxation between the toxin and the bMR (ΔMRₜₒₓᵢₙ). (c) Graphical representation of the K_{D} assay and formula used to calculate the changes in magnetic relaxation when the competitor is competing for binding to the toxin with the bMR (ΔMR_{competitor}).

FIG. 3 shows a representative FTIR spectra for the N₃ modification of a member of the small-molecule library (sulindac). All of the selected molecules displayed the appearance of the 2100 cm⁻¹ stretching band of the N₃ after the azide-linker was chemically coupled to the small molecule. This was indicative of the successful modification needed to attach the small molecule to the nanoparticle.

FIGS. 4A, B, C, D, E, and F show the results of incubating the bMR-nanosensors in the presence of LF with and without competition. Top: Screening results of the detection of LF using bMR nanosensors via magnetic relaxation with three distinct molecules (a) Sulindac, (b) Naproxen, and (c) Fusaric acid. Bottom: The binding of these molecules was confirmed by measuring the dissociation constant between LF and the three distinct molecules (d) Sulindac, (e) Naproxen, and (f) Fusaric acid identified from the screening of the small-molecule library.

FIG. 5 is a line graph showing competitive binding of Sulindac-bMR nanosensors (▴) to LF in the presence of either free Fusaric Acid ( ) or free Naproxen ().

FIGS. 6A, B, C, D, E and F are graphs showing enzymatic activity or cell viability. Top: LF protease activity inhibition studies using (a) Sulindac, (b) Fusaric Acid, and (c) Naproxen as inhibitors. Bottom: LF cytotoxicity inhibition studies using RAW 264.7 cells and (d) Sulindac, (e) Fusaric Acid, and (f) Naproxen as inhibitors. The corresponding IC₅₀ values for each condition were calculated and are herein reported.

FIGS. 7A-F are studies using sulindac metabolic products. Studies using sulindac metabolic products. Top: Computational docking predictions of the binding interaction of LF with (a) Sulindac Sulfide and (b) Sulindac Sulfone. Middle: LF protease activity inhibition studies using (c) Sulindac Sulfide and (d) Sulindac Sulfone. Bottom: LF cytotoxicity inhibition studies using (e) Sulindac Sulfide and (f) Sulindac Sulfone.

FIG. 8 shows small molecules selected with their corresponding structures and classification by FDA status or use.

FIGS. 9A-F shows the chemical structures of (a) Sulindac (b) Fusaric Acid and (c) Naproxen and their predicted binding sites on LF, (d) Sulindac (e) Fusaric Acid and (f) Naproxen via computational docking studies.

FIGS. 10A-D show data of inhibition studies involving chemically modified versions of sulindac and fusaric acid. Left: IC50 measurements of Sulindac-N3 using (a) the fluorogenic substrate assay and (c) the cell viability assay. Right: IC50 measurements of Fusaric Acid-N3 using (b) the fluorogenic substrate assay and (d) the cell viability assay.

FIGS. 11A-D are graphs showing (top) Inhibitory capability of measured using the fluorogenic substrate assay of (A) Sulindac-bMR and (B) Fusaric acid-bMR. (Bottom) Inhibition profiles of (C) Sulindac-bMR and (D) Fusaric acid-bMR against LF using RAW 264.7 cells

FIG. 12 shows magnetic (bMR) nanosensors to screen a library of small molecules for binding to and inhibition of the Anthrax lethal factor (LF). Out of 30 different bMR nanosensors, only two, containing sulindac and fusaric acid on their surfaces, were able to inhibit the protease activity of LF. Meanwhile, the sulindac bMR nanosensor by itself was a potent inhibitor of LF macrophage cytoxocity with an IC50 in the low nanomolar range

### DETAILED DESCRIPTION

Disclosed methods and compositions may be understood more readily by reference to the following detailed description of particular embodiments and the Example included therein and to the Figures and their previous and following description.

### A. METHODS OF TREATING ANTHRAX

### 1. Anthrax

Although both cutaneous and inhalation anthrax can occur, the severity of the inhalation anthrax tends to draw more attention. The anthrax toxins are seen in inhalation anthrax and, not wishing to limit the invention to only inhalation anthrax when compositions and methods may be effective for an anthrax infection (cutaneous or inhaled) treatment, and for brevity, the disclosure herein will refer to compositions and methods for inhibiting anthrax toxin in relation to inhalation anthrax.

In inhalation anthrax, Bacillus anthracis spores are inhaled, giving rise to systemic organ failure within a couple of days. The disease propagates via the release of bacterial spores that can be naturally found in animals or can be weaponized and intentionally released into the environment, similar to the 2001 anthrax letter attacks. Once inhaled, the anthrax spores enter the blood stream where they start to reproduce and release the anthrax toxins, which consist of the lethal factor (LF), protective antigen (PA) and edema factor (EF). These three components work together to affect the host cells, particularly peripheral macrophages, which results in the development of the disease. The killing of the host's macrophages starts when six PA molecules bind to receptors on the surface of the macrophage forming a heptameric subunit that then binds to the LF and EF allowing the endocytic uptake of both subunits and eventual translocation from the endosome into the cytoplasm. Once inside the cell, EF, an adenylate cyclase, elevates cAMP concentration to pathological levels. LF, a Zn-metaloprotease, cleaves the N-terminus of mitogen activated protein kinase kinase (MAPKK), interfering with various signaling pathways. Both mechanisms eventually lead to macrophage death. Out of the two internalized factors, LF has been identified to play a critical role in cell death and studies in animals have shown that mice infected with an anthrax strain lacking LF survive the infection. Furthermore, animal injections of a combination of PA+LF (known as lethal toxin, LeTx) induce a vascular collapse similar to that observed during anthrax infections, pointing to the detrimental effects of LF.

Therefore, disclosed LF inhibitors can provide a strong alternative or combination treatment to the antibacterial treatments commonly used to treat anthrax.

### 2. Methods of Treating

Disclosed are methods of treating a subject having anthrax comprising administering a therapeutically effective amount of one or more carboxylic acid-containing small molecules to the subject, wherein the effective amount of the carboxylic acid-containing small molecule reduces or inhibits lethal factor protease activity.

Carboxylic acid-containing small molecule include, but are not limited to, Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, derivatives thereof, or mixtures thereof. Known carboxylic acid-containing small molecules that have previously been FDA-approved for an indication other than treating anthrax can be used in disclosed methods. FDA-approved carboxylic acid-containing small molecules are known in the art or can be easily determined.

In an aspect, a carboxylic acid-containing small molecule can be sulindac or a derivative thereof. In particular, the sulindac derivative can be a metabolic derivative such as sulindac sulfide or sulindac sulfone.

In an aspect, the carboxylic acid-containing small molecule can be fusaric acid or a derivative thereof.

Disclosed methods of treating a subject having anthrax can be used for treating subjects having inhalation anthrax. Because inhalation anthrax involves the transfer of anthrax spores internally into a subject, the toxins from the spores are released into the blood stream and can lead to lethal consequences if not quickly treated. Therefore, treating subjects having inhalation anthrax may comprise targeting the anthrax toxins and the bacteria itself.

Methods of treating subjects having anthrax may comprise administering a therapeutically effective amount of one or more carboxylic acid-containing small molecules to the subject. A subject having anthrax can be a subject infected with anthrax. In some aspects, a subject can be a subject that is at risk of being exposed to anthrax.

Also disclosed are methods of prophylactic treatment, comprising treating a subject at risk of being exposed to anthrax comprising administering a therapeutically effective amount of a composition comprising one or more carboxylic acid-containing small molecules to the subject, wherein the effective amount of the composition comprising a carboxylic acid-containing small molecule that reduces or inhibits lethal factor protease activity when the subject is exposed to anthrax. The carboxylic acid-containing small molecules disclosed herein can be used.

### 3. Combination Therapies

Methods of treating anthrax can comprise administering an antibacterial compound, wherein the antibacterial compound inhibits or kills the bacteria that causes anthrax. This treatment method allows for the bacteria that causes anthrax to be targeted as well as the lethal factor toxin produced by the bacterial spores to be targeted. Antibacterial compounds that target the bacteria, Bacillus anthracis, that causes anthrax include but are not limited to antibiotics, such as fluoroquinolones (like ciprofloxacin), doxycycline, erythromycin, vancomycin, or penicillin.

Other combination therapies are disclosed. Anthrax can also be treated by administering a therapeutically effective amount of one or more carboxylic acid-containing small molecules, such as sulindac, and a therapeutic agent to a subject in need thereof, wherein the effective amount of the one or morecarboxylic acid-containing small molecules reduces or inhibits lethal factor protease activity. The therapeutic agent may affect the bacteria or one or more of the anthrax toxins, or provide other treatment benefits. For example, sulindac can be administered in combination with raxibacumab, which is a monoclonal antibody that neutralizes anthrax toxins.

The present invention comprises compositions comprising one or more carboxylic acid-containing small molecules and one or more therapeutic agents. Carboxylic acid-containing small molecules include, but are not limited to, Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, Doxorubicin, derivatives thereof, or mixtures thereof. Therapeutic agents include, but are not limited to antibacterial compounds and biological molecules, such as antibodies.

Disclosed combination therapies include the administration of the carboxylic acid-containing small molecule and the antibacterial compound or second therapeutic in any particular order. One or more carboxylic acid-containing small molecule compositions can be delivered before, after or at the same time as a composition comprising one or more therapeutic agents. The different compound or therapeutic compositions can be formulated together or separately. The amount of time between the administration of the different compositions can vary from minutes to hours to days. Compositions may be administered simultaneously or sequentially.

### B. METHODS OF INHIBITING ANTHRAX LETHAL FACTOR

Disclosed are methods of inhibiting anthrax LF toxin activity comprising administering an effective amount of one or more carboxylic acid-containing small molecule compositions to a subject in need thereof LF toxin activity can include metalloproteinase activity that cleaves members of the MAPKK family. The cleavage of the MAPKK signaling protein by LF toxin blocks the signal from the MAPKK signaling protein that recruits immune cells to fight an infection. Therefore, inhibiting anthrax LF toxin activity, or reducing LF protease activity, can lead to an increase in immune cells that are able to respond to the bacterial infection. Carboxylic acid-containing small molecules can include nanoparticles conjugated to carboxylic acid-containing small molecules. Thus, methods of inhibiting anthrax LF toxin activity can include administering an effective amount of a nanoparticle conjugated to a carboxylic acid-containing small molecule in subject in need thereof.

A carboxylic acid-containing small molecule of disclosed methods includes, but is not limited to, Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, derivatives thereof or mixtures thereof.

In an aspect, the carboxylic acid-containing small molecule can be sulindac or a derivative thereof A sulindac derivative can be a metabolic derivative such as sulindac sulfide or sulindac sulfone. In an aspect, the carboxylic acid-containing small molecule can be fusaric acid or a derivative thereof.

Methods of inhibiting anthrax LF toxin activity may comprise administering an effective amount of one or more carboxylic acid-containing small molecules to a subject in need thereof, wherein the carboxylic acid-containing small molecule binds to LF present in the subject. In some aspects, the binding occurs at the active site or the catalytic center of LF. In some aspects, the carboxylic acid-containing small molecule is administered as a nanoparticle- carboxylic acid-containing small molecule conjugate.

Methods of inhibiting anthrax LF toxin activity may comprise administering an effective amount of a carboxylic acid-containing small molecule to a subject in need thereof A subject in need thereof can be a subject infected with anthrax. In some aspects, the subject in need thereof can be a subject that is at risk of being exposed to anthrax.

### C. METHODS OF INHIBITING ANTHRAX TOXIN-INDUCED CYTOTOXICITY

Disclosed are methods of decreasing or inhibiting anthrax toxin-induced cytotoxicity comprising administering an effective amount of a carboxylic acid-containing small molecule to a subject in need thereof. Anthrax toxins cause cell death in the cells that the toxins enter. The PA toxin binds to a cell surface receptor and aides in the binding and internalization of LF toxin. Once internalized, LF contributes to the attack of the cellular machinery of the host cell which ultimately leads to cell death or cytotoxicity. Macrophages can also phagocytose the toxins resulting in cell death of the macrophage. Thus, in some aspects, the methods of decreasing or inhibiting anthrax toxin-induced cytotoxicity involving administering an effective amount of a carboxylic acid-containing small molecule to a subject in need thereof results in reduced macrophage cytotoxicity.

Carboxylic acid-containing small molecules of disclosed methods include, but are not limited to, Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, derivatives thereof, or mixtures thereof.

In an aspect, a carboxylic acid-containing small molecule can be sulindac or a derivative thereof. A sulindac derivative can be a metabolic derivative such as sulindac sulfide or sulindac sulfone. In an aspect, the carboxylic acid-containing small molecule can be fusaric acid or a derivative thereof.

Disclosed are methods of decreasing or inhibiting anthrax toxin-induced cytotoxicity involving administering an effective amount of a carboxylic acid-containing small molecule to a subject in need thereof, wherein the subject in need thereof can be a subject infected with anthrax. In some aspects, the subject in need thereof can be a subject that is at risk of being exposed to anthrax.

### D. METHODS OF ADMINISTRATION

Carboxylic acid-containing small molecule compositions disclosed herein can be administered before, during or after the onset of symptoms associated with anthrax. Any acceptable method known to one of ordinary skill in the art can be used to administer the disclosed carboxylic acid-containing small molecule compositions to a subject.

The administration can be localized (i.e., to a particular region, physiological system, tissue, organ, or cell type) or systemic. The carboxylic acid-containing small molecule compositions can be administered by different routes, such as oral, parenteral and topical. The carboxylic acid-containing small molecule compositions can also be administered directly or indirectly to the site of infection. The particular route of administration selected will depend upon factors such as the particular composition, the severity of the state of the subject being treated, and the dosage required to induce an effective response, such as inhibition of anthrax toxin.

In a preferred embodiment, the carboxylic acid-containing small molecule compositions are administered orally. Effective oral dosages of carboxylic acid-containing small molecule range from about 50 mg to about 250 mg, typically about 150 mg depending on the age of the subject and their kidney function.

An effective level of the carboxylic acid-containing small molecule composition may be reached after one single administration. In certain aspects, administering may comprise two or more doses of carboxylic acid-containing small molecule compositions.

### E. METHODS OF SCREENING

Disclosed are nanoparticle-based screening methods that assess molecular interactions by measuring changes in magnetic relaxation upon ligand binding. Methods of screening comprise a) conjugating a magnetic relaxing (MR) nanosensor to a ligand to form a ligand-MR nanosensor complex; b) contacting the ligand-MR nanosensor complex with a sample containing possible ligand targets; and c) determining the magnetic resonance in the sample, wherein a change in magnetic resonance is indicative of a target bound to the ligand-MR nanosensor complex. In some instances, the change can be an increase in magnetic resonance. In some instances the change can be a decrease in magnetic resonance.

Disclosed ligands that are conjugated to the MR nanosensor can be ligands that are known compounds. For example, a ligand can be an FDA-approved compound or molecule that was FDA-approved for an indication other than treating anthrax.

In an aspect, the ligand can be a carboxylic acid-containing molecule, or one or more carboxylic acid-containing small molecules. For example, the ligands can be, but are not limited to, Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, derivatives thereof, or mixtures thereof.

In an aspect, disclosed methods involve ligands that are FDA-approved drugs. For example, the ligand can be sulindac, fusaric acid, or a derivative thereof. In particular, the sulindac derivative can be a metabolic derivative such as sulindac sulfide or sulindac sulfone.

Disclosed screening methods comprise use of a MR nanosensor that comprises an iron oxide nanoparticle. In an aspect, the iron oxide nanoparticle can be coated with polyacrylic acid.

In an aspect, the screening method comprises contacting the ligand-MR nanosensor complex with a sample containing possible ligand targets, wherein the ligand target can be anthrax toxin. For example, the anthrax toxin can be LF.

Disclosed screening method may comprise a competition assay wherein a small molecule (i.e. a ligand) and a magnetic relaxation nanosensor conjugated with the same small molecule compete for binding to a particular target protein. The magnetic relaxation changes produced by the competitive binding produce a sigmoidal signal response from which the dissociation constant (K_{D}) can be calculated. Using this assay, the binding constants of different interactions between several molecules and macromolecules can be accurately measured. This assay can be used to identify the interaction of compounds or molecules with the anthrax LF toxin, which can then be utilized as LF inhibitors.

1. Magnetic Relaxation Nanosensor

MR nanosensors of disclosed methods can be composed of polymer-coated iron oxide nanoparticles. Other nanoparticles can be but are not limited to gold, silver, fullerene, cerium oxide, gadolinium oxide, carbon nanotube, and polymeric nanoparticles. The nanoparticles can be coated with polyacrylic acid, dextran or any other polymer that facilitates conjugation to a carboxylic acid molecule.

2. Ligand-MR Nanosensor Complex

Disclosed MR nanosensors can be coupled or conjugated to one or more ligands. A ligand can be known compounds, such as carboxylic acid-containing small molecules. In an aspect, the carboxylic acid-containing small molecules have been FDA-approved for an indication other than treating anthrax.

Conjugation can occur between a carboxylic acid of a small molecule and the polyacrylic acid of the nanoparticle. Any conjugation chemistry that involves not only carboxylic acid but aminated nanoparticles can be used. For example, click chemistry can be used.

### F. COMPOSITIONS

The following delivery systems are representative of compositions for administering one or more carboxylic acid-containing small molecule, or one or more carboxylic acid-containing small molecules and one or more therapeutic agents. Compositions disclosed herein may be pharmaceutical compositions, for example, comprising one or more carboxylic acid-containing small molecules and a pharmaceutically acceptable carrier or solution.

1. Parenteral Compositions

Injectable drug delivery systems include pharmaceutically acceptable solutions, suspensions, gels, microspheres and implants. Typically these will be in the form of distilled water, phosphate buffered saline, or other vehicle for injection intravenously or subcutaneously.

2. Enteral Compositions

Oral delivery systems include solutions, suspensions, and solid dosage forms such as tablets (e.g, compressed tablets, sugar-coated tablets, film-coated tablets, and enteric coated tablets), capsules (e.g., hard or soft gelatin or non-gelatin capsules), blisters, and cachets. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). The solid dosage forms can be coated using coatings and techniques well known in the art.

Oral liquid dosage forms include solutions, syrups, suspensions, emulsions, elixirs (e.g., hydroalcoholic solutions), and powders for reconstitutable delivery systems. The compositions can contain one or more carriers or excipients, such as suspending agents (e.g., gums, zanthans, cellulosics and sugars), humectants (e.g., sorbitol), solubilizers (e.g., ethanol, water, PEG, glycerin, and propylene glycol), surfactants (e.g., sodium lauryl sulfate, Spans, TWEENs, and cetyl pyridine), emulsifiers, preservatives and antioxidants (e.g., parabens, vitamins E and C, and ascorbic acid), anti-caking agents, coating agents, chelating agents (e.g., EDTA), flavorants, colorants, and combinations thereof

3. Topical Compositions

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

### G. KITS

The materials described above as well as other materials can be packaged together in any suitable combination as a kit useful for performing, or aiding in the performance of, disclosed methods. It is useful if the kit components in a given kit are designed and adapted for use together in disclosed method. For example disclosed are kits for screening for compounds that bind a target such as anthrax toxin, the kit comprising known compounds and MR nanosensors. The kits also can contain reagents for conjugating the compounds to the MR nanosensors. The kits may comprise instructions for using the components of the kit.

### H. DEFINITIONS

It is understood that disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a carboxylic acid-containing small molecule” includes a plurality of such small molecules, reference to “the magnetic relaxing (MR) nanosensor” is a reference to one or more MR nanosensor and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, “sulindac” refers to sulindac, both R- and S-epimers, sulindac derivatives, metabolites, analogues and variants thereof. Examples of sulindac metabolites include sulindac sulfide, sulindac sulfone. Pharmaceutically acceptable salts are also contemplated. As used herein, “pharmaceutically acceptable salts” refer to derivatives of Disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the sulindac individually. The sulindac may be in the free acid or base form or be pegylated for long acting activity.

The term “anthrax toxin-induced cytotoxicity” refers to the toxic effect of anthrax toxin to cells. The cytotoxicity can result in cell death caused by an anthrax toxin.

As used herein, the term “effective amount” refers to the quantity of a composition which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to reduce or inhibit lethal factor protease activity or anthrax cytotoxicity. The specific effective amount or therapeutically effective amount will vary with such factors as the particular condition being treated, the physical condition of the patient, the type of mammal or animal being treated, the duration of the treatment, the nature of concurrent therapy (if any), and the specific compositions employed and the structure of the compounds or its derivatives.

As used herein, the term “treat” or “treating” refers to partially or completely alleviating, ameliorating, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “subject” or “patient” refers to any organism to which a composition of this invention may be administered, e.g., for experimental, diagnostic, and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

The term “carboxylic acid-containing small molecule” or the like terms refer to a molecule having one or more carboxyl groups or salts thereof and wherein the molecule has a molecular weight of less than 800 g/mole.

Disclosed are materials, compositions, and components that can be used for, can be used in conjunction with, can be used in preparation for, or are products of Disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a carboxylic acid-containing small molecule is disclosed and discussed and a number of modifications that can be made to a number of molecules including the carboxylic acid-containing small molecules are discussed, each and every combination and permutation and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using Disclosed compositions. Thus, if there are a variety of additional steps that can be performed it is understood that each of these additional steps can be performed with any specific embodiment or combination of embodiments of Disclosed methods, and that each such combination is specifically contemplated and should be considered disclosed.

Unless defined otherwise, all technical and scientific terms used herein have the same meanings as commonly understood by one of skill in the art to which Disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize, or be able to ascertain using no more than routine experimentation, many equivalents to the specific embodiments of the method and compositions described herein. Such equivalents are intended to be encompassed by the following claims.

### EXAMPLES

### I. Identification of Sulindac and its Metabolic Derivatives as Inhibitors of Anthrax Lethal Factor

1. Introduction

Magnetic nanosensors have been developed to detect a wide range of molecular targets, achieving unprecedented sensitivity and detection speed. Among those, iron oxide nanoparticle-based magnetic relaxation nanosensors, with the ability of affecting the relaxation times of neighboring water protons have been used to develop multiple sensing technologies. The development of an assay which uses binding magnetic relaxation (bMR) nanosensors to assess molecular interactions by measuring changes in magnetic relaxation upon ligand binding was reported. In this assay, a small molecule and a magnetic relaxation nanosensor conjugated with the same small molecule (bMR nanosensor) compete for binding to a particular target protein in solution or cellular receptor in cultured cells. The magnetic relaxation changes produced by the competitive binding produce a sigmoidal signal response from which the dissociation constant (KD) of that particular interaction can be calculated. Using this assay, the binding constants of different molecular interactions of biological importance, such as avidin-biotin, antibody-antigen and various small molecule-cellular receptors, were measured. This assay can be used to identify small molecule inhibitors of a particular bacterial toxin from a small library of compounds. Particularly, as the assay is done while the small molecule is attached to the nanosensor, the nanosensor itself can also be used as a potential inhibitor of the bacterial toxin. The multivalent display of these small molecule inhibitors on the nanoparticle surface can increase their inhibitory potency.

For the initial studies, the anthrax lethal toxin was selected as a model system. This toxin is released by the Bacillus anthracis in the bloodstream of the host upon infection, giving rise to systemic organ failure within a couple of days. In addition to the anthrax's lethal factor (LF), two other toxins, the protective antigen (PA) and the edema factor (EF) are released by the anthrax bacterium, working in concert to affect the host cells, particularly peripheral macrophages. The killing of the macrophages starts when seven PA molecules bind to receptors on the macrophage's surface forming a heptameric subunit that then binds to LF and EF allowing the endocytic uptake of both subunits. Once inside the cell, EF, an adenylate cyclase, elevates cAMP concentration to pathological levels. LF, a zinc metalloprotease, cleaves the N-terminus of mitogen activated protein kinase kinase (MAPKK), interfering with various signaling pathways. Both mechanisms eventually lead to macrophage death. Out of the two internalized factors, LF has been identified to play a critical role in cell death and studies in animals have shown that mice infected with an anthrax strain lacking LF survive the infection. Furthermore, animal injections of a combination of PA and LF (known as lethal toxin, LeTx) induce a vascular collapse similar to that observed during anthrax infections, pointing to the detrimental effects of LF.

While antibiotics have proven very efficient in eliminating the bacterial infection, they lack the ability to destroy or inhibit the toxins released by the bacteria. This is a significant problem, as LF can remain active in the body for days after the infection has been eliminated causing further macrophage death. This problem is not unique to Anthrax but is also relevant to other toxin-producing bacterial infections. Therefore, the identification of selective and potent toxin inhibitors that can be used as an effective treatment for the disease is a viable therapeutic approach especially when these inhibitors are combined with antibiotics. Several inhibitors of the enzymatic and pathogenic activity of LF have been identified. In order to identify inhibitors of LF a variety of approaches have been utilized, such as library screenings, Mass Spectroscopy-based mining and scaffold-based NMR searches. Results from these screenings have yielded a variety of novel small molecules that inhibit LF at low micromolar concentrations. Although valuable, these small molecules are of low clinical translation with regards to treating LF, as pharmaceutical companies have a low incentive to spend resources and invest millions of dollars to further develop, test and apply for FDA approval of these drugs candidates, due to the low incidence of inhalation anthrax in the general population. Therefore, it is crucial to identify FDA-approved drugs, which are currently used to treat other conditions, as LF inhibitors.

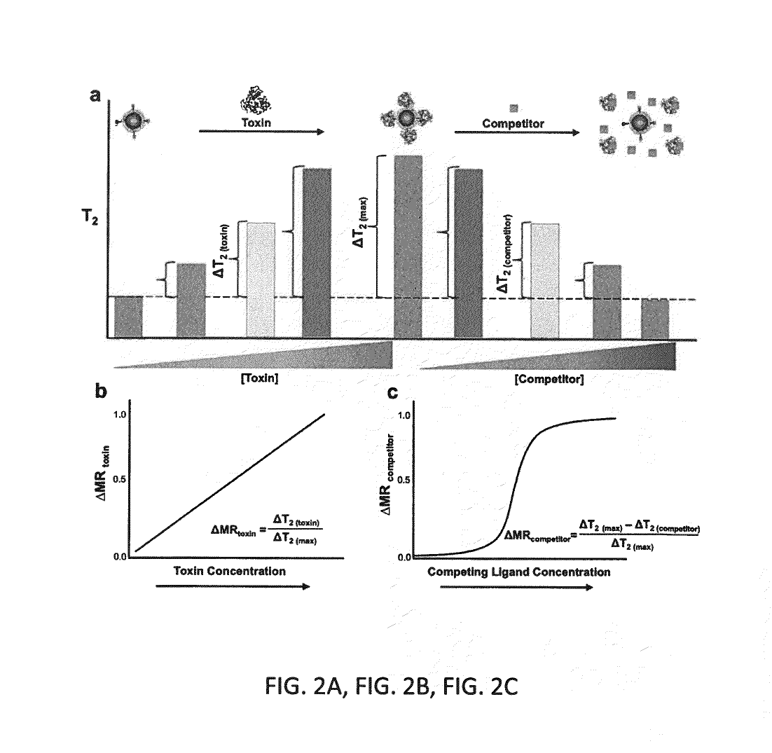

Described herein is the screening with bMR nanosensors of various small molecules, including some FDA-approved drugs, for interaction with and inhibition of LF. A two-part screening method was used to assess and measure the strength of the interaction between the corresponding small molecules and LF (FIG. 2). First, a bMR nanosensor, composed of an iron oxide nanoparticle conjugated to the small molecule of interest, was used to evaluate the binding of this molecule to the toxin. As the bMR nanosensor interacts with increasing concentrations of the LF toxin in solution, the T2 of the sample increases due to the successful binding of the small molecule to LF (FIG. 2). If a potential interaction between LF and a small molecule is identified, the same bMR nanosensor is allowed to compete for binding to LF in the presence of increasing concentrations of the free small molecule in solution (competitor), allowing confirmation of the interaction and estimation of the KD value through magnetic relaxation. The screening identified 3 molecules, sulindac, naproxen and fusaric acid, as compounds that bind to LF with KD values in the micromolar range. Out of these 3 molecules, sulindac, an FDA-approved drug marketed in the USA as Clinoril® for the treatment of pain and swelling associated with osteoarthritis was found to inhibit LF protease activity (IC50=173 μM) and reduced LF cytotoxicity (IC50=31.7 μM) to macrophages. Interestingly, when the corresponding sulindac bMR nanosensor was used as a therapeutic, instead of the free sulindac molecule, a stronger inhibition of the LT proteases activity (IC50=230 nM) and improved macrophage protection (IC50=28.9 nM) were observed. Taken together, these results demonstrate the feasible of the bMR nanosensor method to identify a currently FDA approved drug for the treatment of another disease. Furthermore, our selection process highlights a dependable and translational approach capable of identifying small molecules inhibitors of bacterial toxins.

2. Materials and Methods

i. Materials

All reagents were of analytical reagent grade. The 30 small-molecule members of the library were obtained from Sigma Aldrich and they are: Bezafibrate, Sulindac, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin. Sulindac Sulfide, Sulindac Sulfone, the Iron salts (Fe2Cl3.4H2O and Fe3Cl3.6H2O), Polyacrylic acid (PAA, MW 1.8 kDa), ammonium hydroxide, hydrochloric acid, propargylamine, N-hydroxysuccinimide (NHS), HEPES, TWEEN 20, calcium chloride, and DMSO were also obtained from Sigma Aldrich whereas EDC (1-ethyl-3-[3-dimethylaminopropyl]-carbodiimide hydrochloride) was obtained from Pierce Biotechnology. The Anthrax Lethal Factor was obtained from List Biological Laboratories, INC. The anthrax lethal factor protease substrate III, fluorogenic was obtained from Calbiochem. AutoDock 4.2. AutoDockTools 1.5.4 was downloaded free of charge from the Scripps Research Institute's website (autodock.scripps.edu). RAW 264.7 cells were obtained from ATCC.

ii. Synthesis of bMR Nanosensors

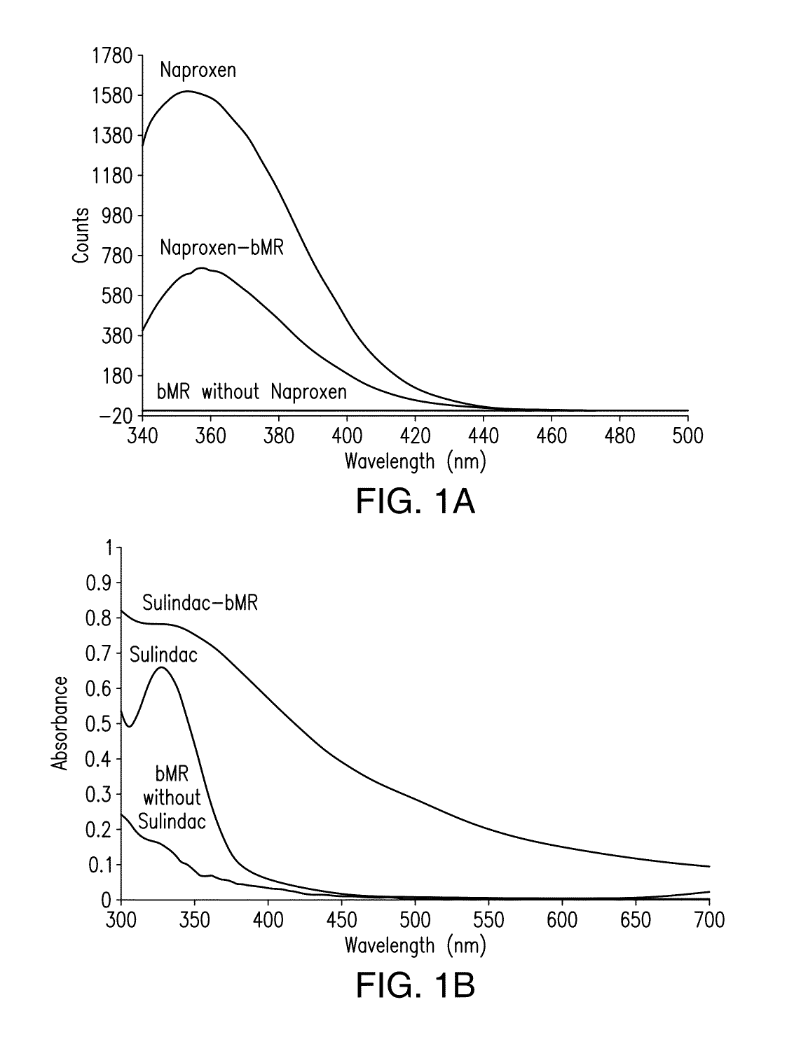

The bMR nanosensors used in these studies are composed of polyacrylic acid coated iron oxide nanoparticles that has been conjugated with the various small molecule drugs following previously described reports²⁹⁻³¹. Briefly, polyacrylic acid coated iron oxide nanoparticles [PAA-IONPs] were derivatized with a propargyl group by incubating the nanoparticles (50 mg Fe) in MES buffer (30 mL, pH=6.5) with a solution of EDC (115.2 mg, 0.6 mmol) and NHS (69 mg, 0.6 mmol) in MES buffer (2.5 mL). To the resulting reaction mixture, propargylamine (33 mg, 0.6 mmol) in DMSO (0.75 mL) was added drop-wise under medium stirring and incubated for 6 h at room temperature. The resulting reaction mixture, containing the propargylated nanoparticles, was then purified using a magnetic column and concentrated using KrosFlow filtration system to approximately 5 mg Fe/ml in PBS. The nanoparticles were stored at 4° C. FT-IR data analysis confirmed the conjugation with the propargyl group by the observing the appearance of the amide N—H bending (1550 cm⁻¹) and C═O stretching (1640 cm⁻¹) as well as the alkyne C≡C stretching at 2260 cm⁻¹. Meanwhile, to conjugate the small molecule drugs to the propargylated nanoparticles, each one of the various small molecules were modified with an azide containing linker (3-azidopropan-1-amine) that was prepared as previously described.³² The corresponding 30 azide-derivatized small molecules were then conjugated to propargylated iron oxide nanoparticles, yielding 30 different bMR nanosensors. In a typical procedure, propargylated poly(acrylic acid)-coated iron acid nanoparticles (3 mg, 2 mg/mL, 1 equiv. in NaHCO₃ buffer pH. 8.5) were added to each one of the azide-functionalized small molecules (5 equiv. in DMSO) The reaction was initiated at room temperature in the presence of catalytic amount of CuI (0.01 μg in 500 μL of bicarbonate buffer, pH 8.5), and was further incubated for 12 h at room temperature. The final reaction mixture was purified with a magnetic column (LS25, Miltenyi) using DMSO as the elutant. The small molecule-carrying nanoparticle preparations were stored in DMSO at room temperature until further use. Confirmation of the successful conjugation of the small molecules to the nanoparticles was achieved through either UV-Vis absorption spectroscopy or Fluorescence spectroscopy, depending on the spectroscopic profile of each individual molecule (FIG. 1).

iii. Assay for the Screening of the Small Molecule Library Against LF

bMR nanosensor solutions, consisting of 10 μL (3 mg/mL) of the bMR-nanosensors and 2,000 μL DI water was prepared. Samples containing different concentrations of the LF toxin (2 μM to 20 nM) in 1×PBS buffer were prepared and 2 μL of each sample was added to 200 μL of the bMR analyzing solution. A negative control sample was prepared in the same fashion, adding 2 μL fresh 1×PBS buffer instead of toxin. Magnetic relaxation measurements were performed every 15 minutes of incubation at room temperature for 1 hour. Transverse (T₂) proton relaxation times measurements were obtained using a Bruker Minispec mq20 NMR analyzer operating at a magnetic field of 0.47 T and at 37° C. The T₂ from each individual toxin sample was subtracted from that of the control to calculate the ΔT₂₍ₜₒₓᵢₙ₎. This was then divided by the largest possible ΔT₂₍ₘₐₓ₎ in order to obtain a binding percentage denoted as ΔMRₜₒₓᵢₙ (FIG. 2) The ΔMRₜₒₓᵢₙ was plotted against the LF concentrations in order to access the binding of the bMR nanosensor. In order to calculate the K_{D} of the discovered interactions we used a previously published assay. A small-molecule(SM)-carrying bMR was utilized at a concentration of 0.015 mg Fe/mL and an analyzing solution was prepared that consisted of 4.5 μL SM-carrying bMRs and 2,000 μL of de-ionized water. Samples containing 10 μL of different concentrations of free-SM (competing ligand 0.05-500 μM, in DI water) and 200 μL of the SM-bMR analyzing solution (bMR nanosensor) were prepared and incubated with 10 μL of LF (1 nM). A negative control sample was prepared in the same fashion, adding 10 μL of DI water instead of free-SM (0 M free-SM control sample). Magnetic relaxation measurements were performed after 15 minutes of incubation at room temperature. Transverse (T₂) proton relaxation times measurements were done using a Bruker Minispec mq20 NMR analyzer operating at a magnetic field of 0.47 T and at 37° C. The MR_{(competitor)} value was calculated using the [ΔT₂ (max)−ΔT_{2(competitor)}]/ΔT₂₍ₘₐₓ₎ formula, in which ΔT₂₍ₘₐₓ₎ refers to the change in T₂ when there is not competitor present, and ΔT_{2(competitor)} represents the change in T₂ when each concentration of the competitor is added.

iv. Blind Docking Studies

Blind docking studies were performed using the default parameters in AutoDock 4.2. Briefly, a three-dimensional grid that covered all the atoms of the LF was prepared. This cubed shaped grid measured 126,90,114 points in the xyz planes and had a spacing of 0.78611 Å. After the affinity maps were generated from the grid, the docking search was performed using AutoDock's Lamarckian genetic algorithm on its default settings. The logs containing the results from the docking experiments were analyzed and visualized using AutoDockTools.

v. Competitive Binding Experiment

In order to perform this experiment a working solution of the Sulindac-bMR (0.015 mg/mL) containing 2.5 mM of fusaric acid or naproxen was used and it was incubated with different concentrations of LF (2 pM to 20 nM). The resulting T₂ were measured using a Bruker Minispec mq20 NMR analyzer operating at a magnetic field of 0.47 T and at 37° C. and the corresponding ΔMRₜₒₓᵢₙ values calculated.

vi. Anthrax Lethal Factor Protease Inhibition Assay, General Procedure

In a fluorescence (black) 96-well plate, samples (100 μL) containing LF (2 nM) in a 40 mM HEPES at pH 7.2, 100 μM CaCl₂, 0.05% (v/v) buffer and different concentrations of inhibitors (Table 1) were prepared and incubated for 30 minutes. After the incubation period, 0.5 μL of fluorogenic anthrax lethal factor protease substrate III (470 μM, DMSO) was added to each sample and the fluorescence was measured every 10 minutes for an hour using a Tecan infinite M200 at a 355 nm excitation and 460 nm emission. A control sample was prepared using water instead of small molecule inhibitor. IC₅₀ concentrations were calculated from the data collected by plotting the ratio of the fluorescence from each sample to that of the control versus the concentration of the inhibitor.

TABLE 1
Concentration range of the inhibitors used
for the ALF protease inhibition assay.

| Inhibitors | Concentration Range | |
|---|---|---|
| Sulindac | 0.005-5 | mM |
| Fusaric Acid | 0.1-10 | mM |
| Naproxen | 0.1-10 | mM |
| Sulindac Sulfide | 0.010-1 | mM |
| Sulindac Sulfone | 0.010-1 | mM |
| Sulindac-N₃ | 0.010-20 | mM |
| Sulindac on bMR | 0.020-4 | μM |
| Fusaric Acid-N₃ | 0.050-50 | mM |
| Fusaric Acid on bMR | 0.05-50 | μM |

vii. Anthrax Lethal Factor Inhibition Cell-Viability Assay, General Procedure

Raw 264.7 cells were grown in a high-glucose (4500 mg/L), 10% Fetal bovine Serum (FBS) Dulbecco's Modified Eagle Medium and seeded into a 96-well plate. The cells were then treated with different concentrations of the small-molecule inhibitor (Table 2) and incubated for 1 hr, before being treated with 5 nM LF and 15 nM PA. After a 4-hour incubation, the media was removed and each well was washed three times with 1×PBS before being treated with a MTT solution (2 mg/mL) for 3 hours. The resulting formazan crystals were dissolved in acidified isopropanol, and the absorbance was recorded 570 and 750 nm (background) using a Synergy HT multidetection microplate reader (Biotek). These experiments were performed in triplicate.

TABLE 2
Concentration range of the inhibitors used for the Anthrax
Lethal Factor Inhibition Cell-Viability Assay

| Inhibitors | Concentration Range | |
|---|---|---|
| Sulindac | 1-250 | μM |
| Fusaric Acid | 1-500 | μM |
| Naproxen | 2.5-500 | μM |
| Sulindac Sulfide | 0.5-175 | μM |
| Sulindac Sulfone | 1-250 | μM |
| Sulindac-N₃ | 0.001-0.5 | mM |
| Sulindac on bMR | 0.001-1 | μM |
| Fusaric Acid-N₃ | 0.050-10 | μM |
| Fusaric Acid on bMR | 0.005-0.75 | mM |

viii. LF Protease and Cell Viability Inhibition Assay Using bMR Nanosensors as Inhibitors.

These experiments were carried out as described in the sections above, but instead the bMR nanosensors were used as inhibitors using the calculated amounts of drug on the nanoparticle surface. The concentrations of the corresponding small molecules on the bMRs were calculated using procedures previously described.

3. Results and Discussion

i. Small Molecule Library Selection and Development of Corresponding bMR-Nanosensors

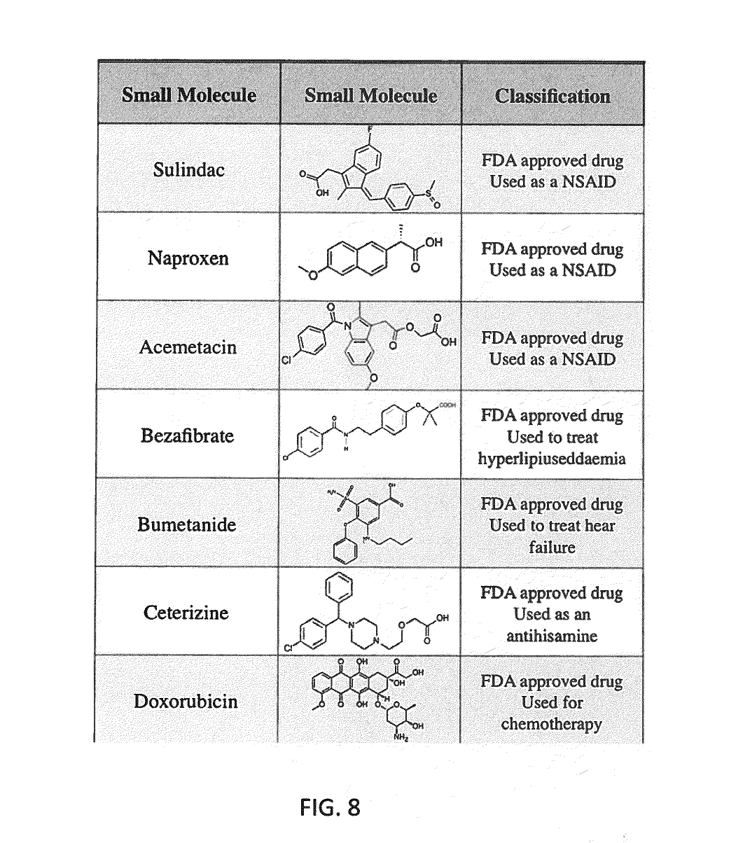

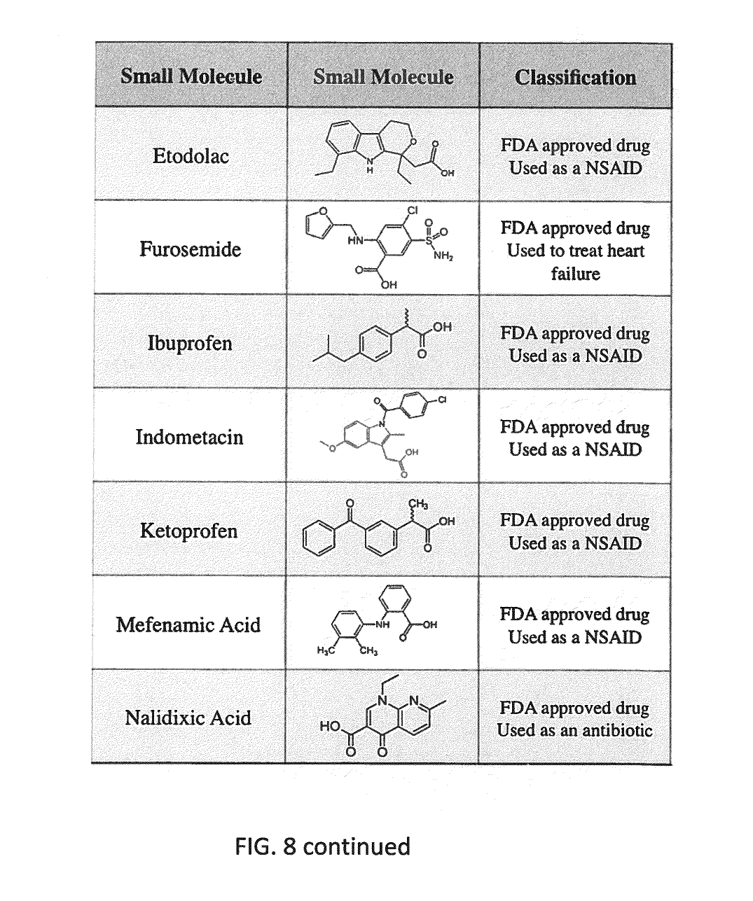

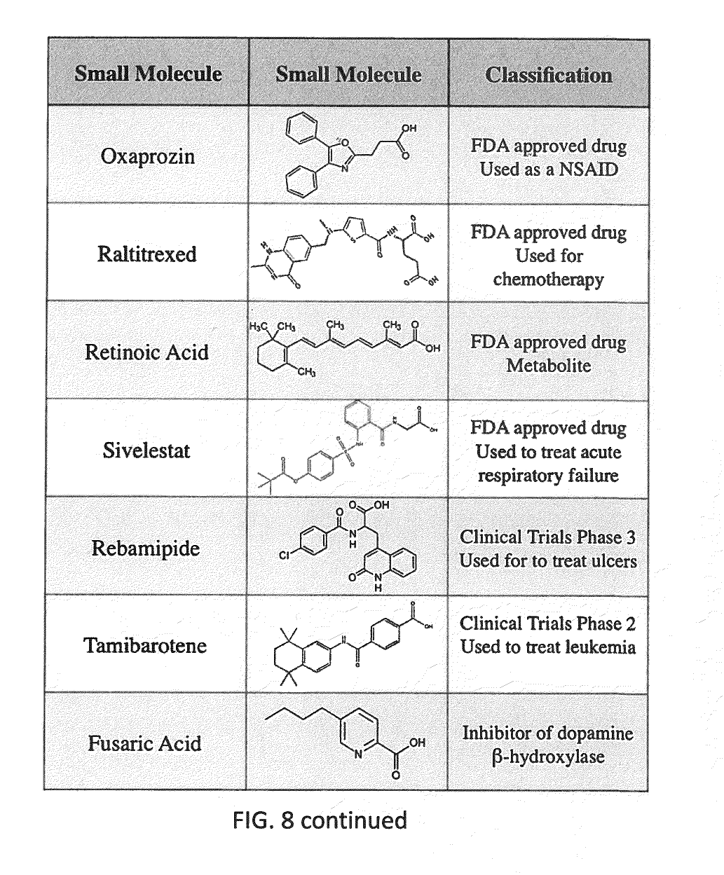

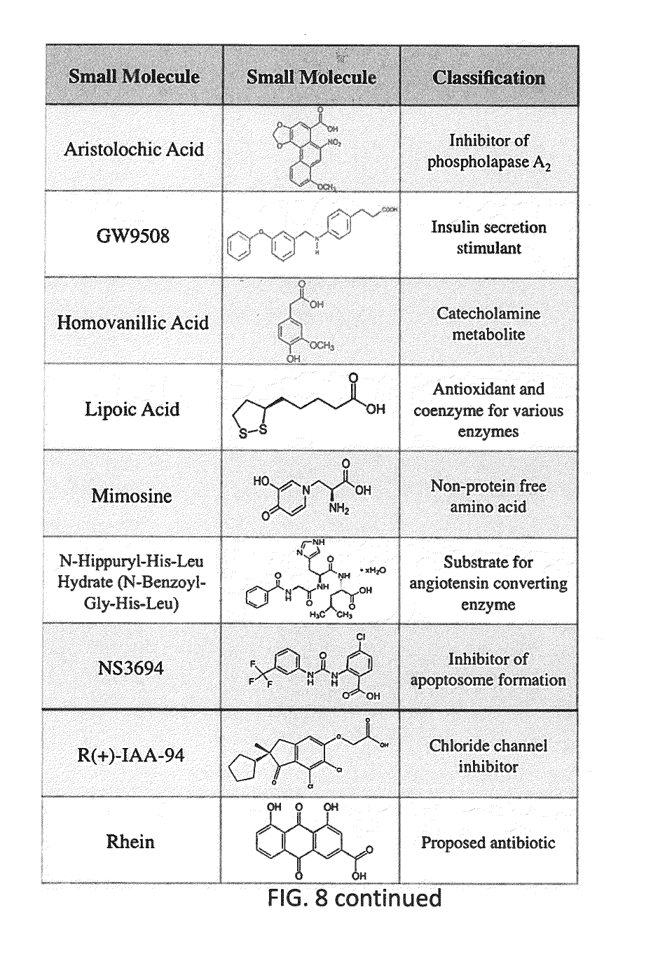

To fabricate a library of bMR nanosensors for the screening of small molecules as binding ligands and potential inhibitors of the Anthrax LF toxin, close to 1,000 commercially available carboxylic-acid-containing small molecules were identified. These molecules were selected specifically with that functional group in order to facilitate their conjugation to the polyacrylic acid coated ion oxide nanoparticles. Out of these 1,000 molecules, 30 molecules were selected based on the following criteria (1) structural similarity to known inhibitors of LF, particularly the presence of multiple 5-carbon and 6-carbon rings, (2) polar aromatic compounds and amphiphilic compounds soluble in DMSO or DMF, (3) cost effective and easily available and (4) either a small molecule which is FDA-approved, is in late clinical trials, or is being studied as a potential drug for another disease (FIG. 8).

To facilitate the conjugation of these small molecules to the polyacrylic acid-coated iron oxide nanoparticles, the small molecules were coupled to an azide linker (3-azide propylamine) through their respective carboxylic acid groups using carbonyldiimidazole chemistry yielding a series of 30 azide-conjugated small molecules (Table 3).

TABLE 3
Reaction details for the coupling of the azide linker to the small molecules

| | | | | | | Wt. Azide | | N₃ IR |
|---|---|---|---|---|---|---|---|---|
| Small Molecule | Solvent | Wt. SM | | Wt. CDI | | Linker | | Band |
| Acetametacin | CHCl₃ | 92.3 | mg | 43.1 | mg | 22.2 | mg | 2096 cm⁻¹ |
| Aristolochic Acid I | THF | 25 | mg | 14.2 | mg | 7.32 | mg | 2097 cm⁻¹ |
| Bezafibrate | THF | 100 | mg | 53.7 | mg | 27.6 | mg | 2096 cm⁻¹ |
| Butanemide | THF | 87 | mg | 46.5 | mg | 23.8 | mg | 2098 cm⁻¹ |
| Ceterizine HCl | THF | 50 | mg | 21.0 | mg | 10.8 | mg | 2097 cm⁻¹ |
| Doxorubicin | THF | 10 | mg | 3.35 | mg | 1.72 | mg* | 2098 cm⁻¹ |
| Etodolac | CH₂Cl₂ | 10 | mg | 6.8 | mg | 3.5 | mg | 2093 cm⁻¹ |
| Furosemide | THF | 99.1 | mg | 58.2 | mg | 30.0 | mg | 2097 cm⁻¹ |
| Fusaric Acid | CHCl₃ | 91.5 | mg | 99.3 | mg | 51.1 | mg | 2093 cm⁻¹ |
| GW9508 | CH₂Cl₂ | 5.0 | mg | 2.8 | mg | 1.7 | mg | 2097 cm⁻¹ |
| Homovanilic Acid | THF | 69 | mg | 73.7 | mg | 37.9 | mg | 2097 cm⁻¹ |
| Ibuprofen | CHCl₃ | 98.3 | mg | 92.6 | mg | 47.6 | mg | 2093 cm⁻¹ |
| Indometacin | CHCl₃ | 100 | mg | 54.3 | mg | 28.0 | mg | 2096 cm⁻¹ |
| Ketoprofen | CHCl₃ | 100 | mg | 76.5 | mg | 39.4 | mg | 2093 cm⁻¹ |
| L-Mimosine | THF | 25 | mg | 24.5 | mg | 12.6 | mg | 2102 cm⁻¹ |
| Lipoic Acid | CH₂Cl₂ | 100 | mg | 94.4 | mg | 48.5 | mg | 2092 cm⁻¹ |
| Mefenamic Acid | CHCl₃ | 100 | mg | 80.7 | mg | 41.5 | mg | 2094 cm⁻¹ |
| N-Hippuryl-His-Leu | THF | 25 | mg | 11.3 | mg | 7.0 | mg | 2097 cm⁻¹ |
| Nalidixic Acid | CHCl₃ | 100 | mg | 83.8 | mg | 43.1 | mg | 2097 cm⁻¹ |
| Naproxen | CHCl₃ | 100 | mg | 84.5 | mg | 43.5 | mg | 2096 cm⁻¹ |
| NS3694 | THF | 5.0 | mg | 2.71 | mg | 1.39 | mg | 2095 cm⁻¹ |
| Oxaprozin | THF | 5.0 | mg | 3.31 | mg | 2.0 | mg | 2094 cm⁻¹ |
| R(+)-IAA-94 | CHCl₃ | 10 | mg | 5.4 | mg | 3.4 | mg | 2096 cm⁻¹ |
| Raltiterexed | THF | 10 | mg | 4.1 | mg | 2.0 | mg | 2099 cm⁻¹ |
| Rebamipide | CH₂Cl₂ | 5.0 | mg | 2.62 | mg | 1.6 | mg | 2098 cm⁻¹ |
| Retinoic Acid | THF | 50 | mg | 32.4 | mg | 16.7 | mg | 2099 cm⁻¹ |
| Rhein | CHCl₃ | 50 | mg | 34.2 | mg | 17.6 | mg | 2096 cm⁻¹ |
| Sivelestat | THF | 5.0 | mg | 2.24 | mg | 1.15 | mg | 2101 cm⁻¹ |
| Sulindac | CH₂Cl₂ | 100 | mg | 54.5 | mg | 28.1 | mg | 2094 cm⁻¹ |
| Tamibarotene | CHCl₃ | 5.0 | mg | 2.8 | mg | 1.7 | mg | 2097 cm⁻¹ |

*A different azide-linker was used, 2-azidopropanoic acid

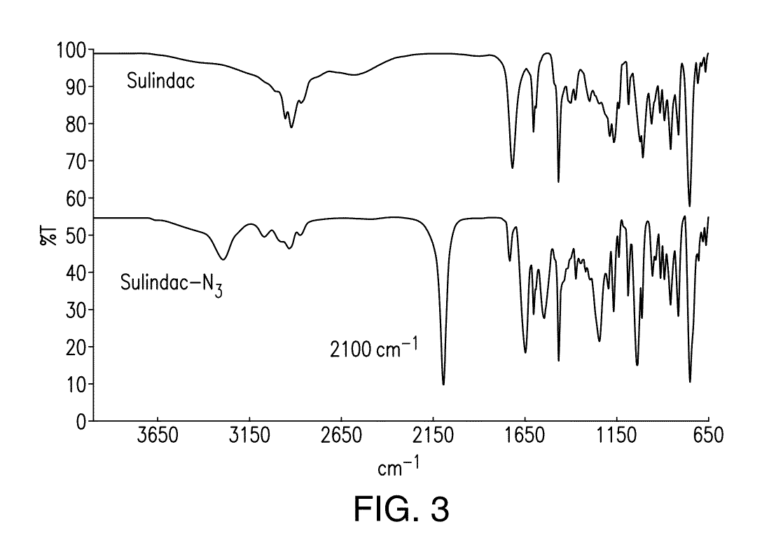

The successful conjugation of an azide group to each of the selected 30 small molecules was verified via FTIR, by monitoring the appearance of a stretching band at 2100 cm⁻¹ characteristic of the N₃ functional group in the azide linker (FIG. 3). The resulting azide-conjugated small molecules were then conjugated to propargyl-derivatized PAA-IONP (75 nm, R₂: 230 mM⁻¹s⁻¹) using click chemistry as previously described. The successful conjugation of the small molecules to the nanoparticle surface was verified by either fluorescence or absorbance spectroscopy, depending on the corresponding physical properties of the molecule. This allowed for the determination of the number of molecules per nanoparticle following previously reported procedures. The IONPs by themselves are not fluorescence and have a very low absorbance, therefore making it easy to distinguish the small molecule on the surface of the IONP (FIG. 1). On average, an iron oxide nanoparticle contained 5-9 small molecules, which corresponds to low valency bMR-nanosensors.

ii. Screening of the bMR-Nanosensors Library for Binding to Anthrax LF Toxin

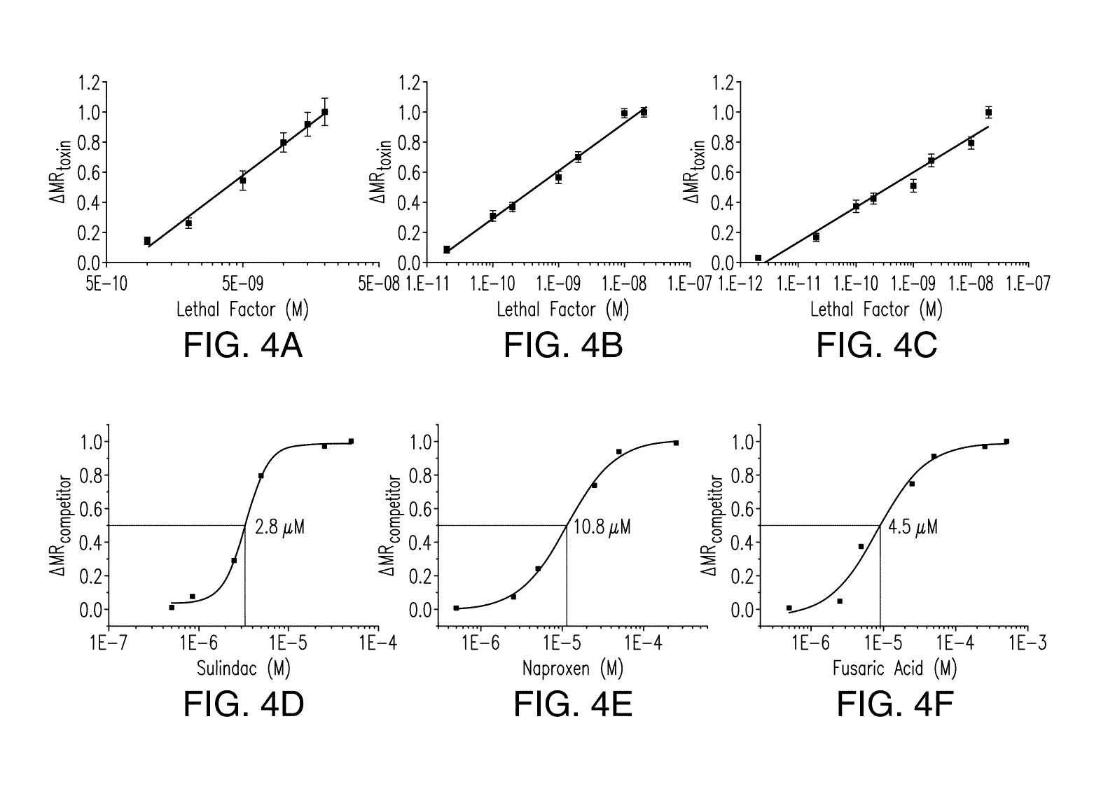

Each member of the bMR-nanosensors library was first tested for binding to LF by incubating the nanosensors [0.15 μg Fe/ml] with increasing concentration of LF toxin [2 pM to 20 nM] in PBS buffer, pH 7.2 for 30 minutes at room temperature. The screening was performed by measuring the changes in T₂ of a suspension of bMR-nanosensors after incubation with increasing concentrations of toxin (ΔT₂₍ₜₒₓᵢₙ₎). The reported ΔMRₜₒₓᵢₙ value is then calculated by dividing the ΔT₂₍ₜₒₓᵢₙ₎ by the ΔT₂ of the highest toxin concentration (ΔT_{2 (max)}) as shown in FIG. 2B. A linear increase in ΔMRₜₒₓᵢₙ signal with increasing concentrations of the toxin (LF), indicates a successful binding interaction between the LF and the corresponding bMR-nanosensors. Results showed that only 3 of the 30 bMR-nanosensors tested displayed an increase in ΔMRₜₒₓᵢₙ signal upon incubation with increasing concentrations of LF (FIG. 4 A-C). These nanosensors were those with either sulindac, naproxen and fusaric acid conjugated on the nanoparticle's surface. These results indicate an interaction of the corresponding small molecules (sulindac, naproxen and fusaric acid) with the anthrax LF toxin while still attached on the nanoparticle surface. Further confirmation of this molecular interaction between LF and the small molecules on the nanoparticle was obtained by measuring the dissociation constant (K_{D}) using a competition magnetic relaxation assay. Briefly, the LF toxin and the bMR-nanosensors were incubated with increasing amounts of the corresponding free small molecules (sulindac, naproxen or fusaric acid) as competitor. The competition of the bMR nanosensor and the increasing amounts of free molecules in solution for binding to the toxin provided us with a method to estimate the K_{D} of the studied interactions (FIG. 4 D-F) following a previously reported method. The corresponding ΔMR_{competitor} values were calculated by first subtracting the changes in T₂ caused by the addition of the competitor (ΔT_{2(competitor)}) from the maximum possible change in T₂ (ΔT₂₍ₘₐₓ₎), which occurs when there is not competitor available. This value is then divided by the ΔT₂₍ₘₐₓ₎ in order to obtain the ΔMR_{competitor}. A sigmoidal response observed when plotting the ΔMR_{competitor} versus increasing concentrations of the corresponding competitor indicates a successful binding interaction and allows for determination of the corresponding K_{D} value (FIG. 2c). The results of the screening are summarized in Table 3, where the successful binding of the small molecule to LF is shown by reporting its K_{D} value.

TABLE 4
Small molecule library screening results. The discovered
interactions are reported as K_{D} values. 1-18 are FDA approved
drugs; 19 and 20 are those clinical trials.

| | | Anthrax Lethal |
|---|---|---|
| | Small Molecule Library | Factor (LF) |
| 1 | Sulindac | K_{d} = 2.8 μM |
| 2 | Naproxen | K_{d} = 10.8 μM |
| 3 | Acemetacin | — |
| 4 | Bezafibrate | — |
| 5 | Bumetanide | — |
| 6 | Ceterizine HCL | — |
| 7 | Doxorubicin | — |
| 8 | Etodolac | — |
| 9 | Furosemide | — |
| 10 | Ibuprofen | — |
| 11 | Indometacin | — |
| 12 | Ketoprofen | — |
| 13 | Mefenamic Acid | — |
| 14 | Nalidixic Acid | — |
| 15 | Oxaprozin | — |
| 16 | Raltiterexed | — |
| 17 | Retinoic Acid | — |
| 18 | Sivelestat | — |
| 19 | Rebamipide | — |
| 20 | Tamibarotene | — |
| 21 | Fusaric Acid | K_{d} = 4.5 μM |
| 22 | Aristolochic Acid I | — |
| 23 | GW9508 | — |
| 24 | Homovanilic Acid | — |
| 25 | L-Mimosine | — |
| 26 | Lipoic Acid | — |
| 27 | N-Hippuryl-His-Leu | — |
| 28 | NS3694 | — |
| 29 | R(+)-IAA-94 | — |
| 30 | Rhein | — |

The results indicated that three small molecules interact strongly with the LF toxin, while attached to a magnetic nanoparticle. Interestingly, two of them Sulindac and Naproxen are FDA-approved non-steroidal anti-inflammatory drugs (NSAIDs) that inhibit the COX(I/II) enzyme (IC50: COX-1=41.3 μM, COX-2 25.0 μM)22. The other molecule that interacted with LF, fusaric acid, is not FDA-approved but it is being studied for various medical applications. Using a competition magnetic relaxation assay, the dissociation constant between LF and the corresponding molecules was estimated (FIG. 1 d-f). Sulindac showed the strongest interaction binding to LF with a KD of 2.8 μM. Fusaric acid was the second strongest binder to LF with a dissociation constant of 4.5 μM, followed by naproxen with a KD of 10.8 μM. These KD values further confirmed that these three molecules strongly interact to the LF toxin while being attached to the nanoparticles. These results also indicate that conjugation of these molecules to the nanoparticle surface does not significantly affect binding of the molecule to the LF toxin.

iii. Competitive Binding Study

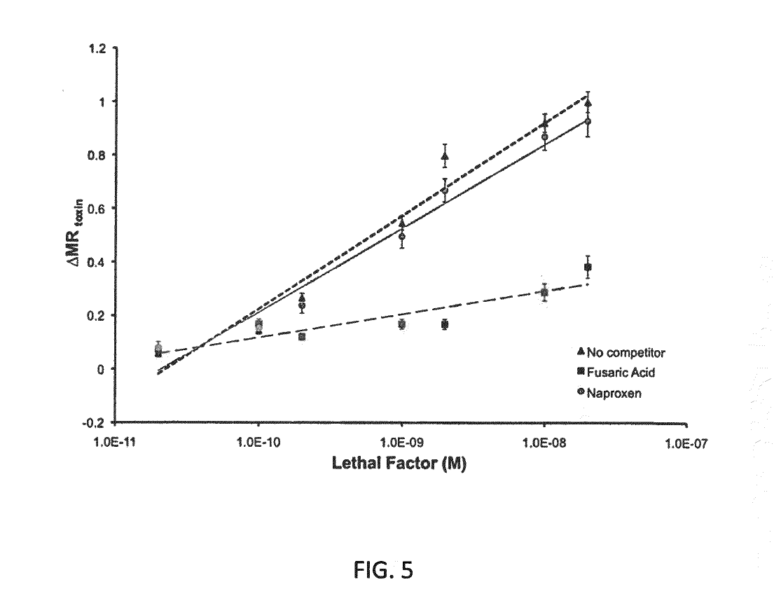

Whether one of these molecules affects the binding of the others to LF was also investigated. For these studies, a competition assay was performed where the binding of one molecule to LF would affect the binding of the other as determined by magnetic relaxation. Specifically, the changes in MRₜₒₓᵢₙ of a suspension of sulindac bMR nanosensor was measured upon binding to increasing concentrations of LF in the presence of a set amount of either fusaric acid or free naproxen as competitor. It is expected that if sulindac and fusaric acid or naproxen bind to the same area within the LF toxin, the free small molecule will compete for binding to LF with the sulindac bMR nanosensor, thus affecting the magnetic relaxation signal of the nanosensors in suspension. When fusaric acid was used as a free competitor, a significant decrease in the ΔMRₜₒₓᵢₙ was observed as the concentration of LF increased, indicating that fusaric acid is interfering with the binding of LF to the sulindac bMR nanosensor (FIG. 5). Instead, when naproxen was used as a competitor, no significant decrease in ΔMRₜₒₓᵢₙ signal was observed, indicating that naproxen does not bind in the same location as sulindac.

iv. Computational Studies

Since the X-ray structure of Anthax LF toxin is known, computer simulation studies were performed with Autodock 4.2 to predict where sulindac, naproxen and fusaric acid most probably bind to LF. Autodock 4.2 is a widely used molecular docking program capable of predicting where a particular molecule binds to a protein. The program searches for the lowest energy binding conformation using a Lamarckian genetic algorithm. This algorithm allows the conformation of the small molecule to change and compete in a manner similar to biological evolution, consequently selecting the conformation with the lowest binding energies. One specific feature that made Autodock useful in these studies was its ability to perform blind-docking studies. Under these studies, the software predicts the binding sites of our small molecules on LF by searching for locations throughout the entire structure of the toxin. This was of particular importance because it facilitated the prediction where each of the small molecules potentially binds to the toxin.

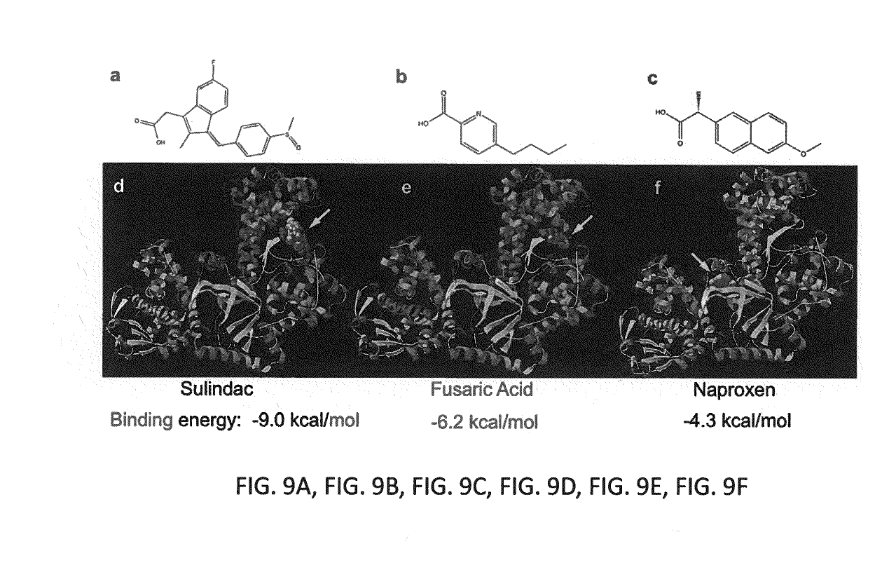

The blind-docking studies were performed using the default parameters in Autodock 4.2. Sulindac was the first molecule studied using this program. Different interactions of sulindac with LF's domains III and IV were observed, with two of the conformations binding around or to the enzymatic pocket, which is located in domain IV. However, the interaction with the lowest binding energy (−9.0 kcal/mol) was that of sulindac with domain IV at the catalytic site of LF (FIGS. 9a and 9d). It is important to point out that these are in silico predictions and without an X-ray structure of the LF toxin in the presence of sulindac, one cannot undoubtedly claim that this is how sulindac binds to LF. However, these predictions can be used as a framework to estimate how this interaction could occur in light of the data presented herein.

Next, fusaric acid was studied, which according to the KD measurements was the molecule with the second strongest binding to LF. As with sulindac, the conformation with the most favorable binding was also observed at the catalytic center in domain IV with a binding energy of −6.2 kcal/mol (FIGS. 9b and 9e). Lastly, docking studies were performed between naproxen and LF. Results from these studies predicted that naproxen would also bind on several locations on the LF, but unlike sulindac and fusaric acid, none of these predictions involved the catalytic center, and instead all the low binding energy conformations involved areas on domains I and II. The conformation with the strongest interaction between naproxen and LF was observed between domains I and II and had a binding energy of −4.3 kcal/mol (FIGS. 9c and 9f). Taken together, these computational docking studies indicate that sulindac strongly binds to LF with the lowest binding energy among the molecules studied (−9.0 kcal/mol), followed by fusaric acid (−6.2 kcal/mol) and naproxen (−4.3 kcal/mol). Interestingly, this order of binding energy correlates with the order of affinities observed in the KD values of sulindac (2.8 μM), fusaric acid (4.5 μM) and naproxen (10.8 μM), as calculated by the bMR nanosensor competition assay. Most importantly, these computer simulation studies indicated that both sulindac and fusaric acid bind to the catalytic center of LF, indicating a potential use as inhibitors of LF's protease activity and cell toxicity.

Since the computational studies predicted that sulindac and fusaric acid bind to the same region on LF, its catalytic center, a competition assay was performed to assess if naproxen can compete for binding with sulindac. Specifically, the changes in MRtoxin of a suspension of sulindac bMR nanosensor upon binding to increasing concentrations of LF in the presence of a set amount of either fusaric acid or free naproxen as competitor were measured. It is expected that if sulindac and fusaric acid bind to the same location within the LF toxin, free fusaric acid will compete for binding to LF with the sulindac bMR nanosensor, thus affecting the magnetic relaxation signal of the nanosensors in suspension. When fusaric acid was used as a free competitor, a significant decrease in the ΔMRtoxin was observed as the concentration of LF increased, indicating that fusaric acid is interfering with the binding of LF to the sulindac bMR nanosensor (FIG. 5). Instead, when naproxen was used as a competitor, no significant decrease in ΔMRtoxin signal was observed, further supporting the findings of the computational studies that naproxen does not bind in the same location as sulindac.

v. Inhibition of LF Protease Activity by Sulindac and Fusaric Acid

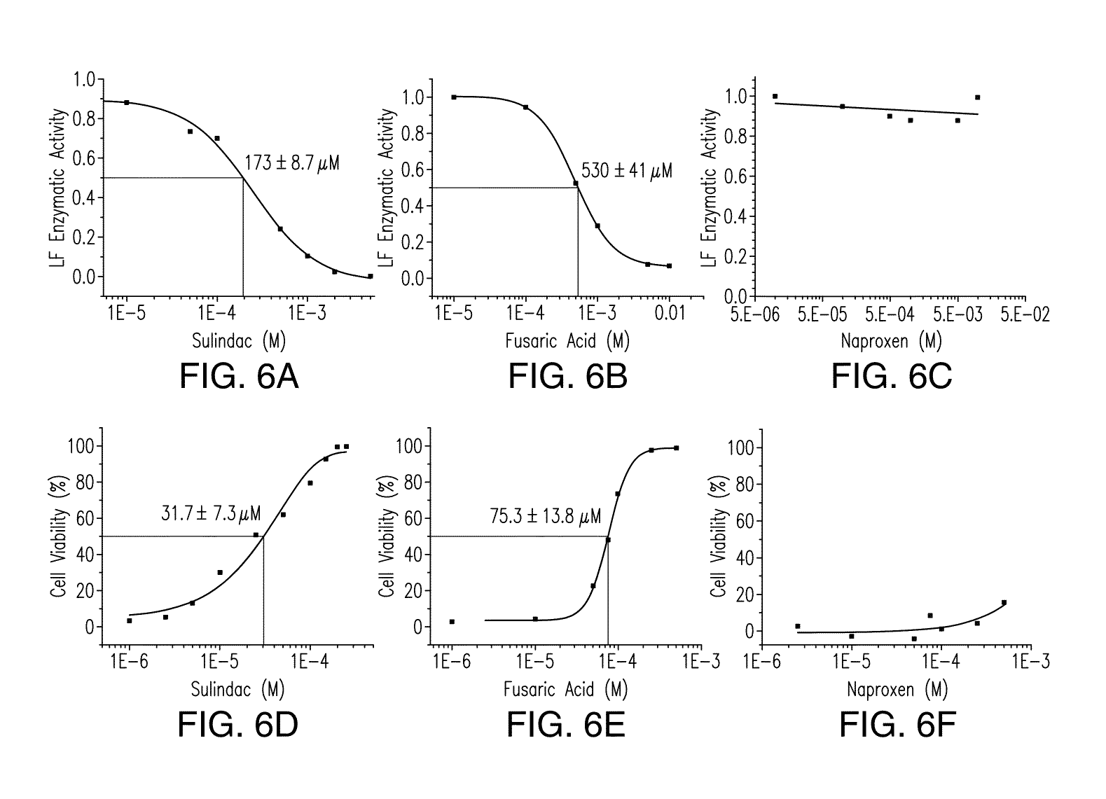

Since the computational docking studies predicted the binding of sulindac and fusaric acid to the catalytic site of anthrax LF toxin, we reasoned that these two molecules could potentially inhibit the protease activity of LF. To examine this hypothesis, we tested the ability of sulindac and fusaric acid to inhibit the protease activity of LF using an activatable fluorogenic substrate. In this assay, a peptide derived from the Mitogen-Activated Protein Kinase Kinase 2 protein (MAPKK2), the natural target for LF, was used. The peptide (Ac-GYβARRRRRRRRVLR-AMC; SEQ ID NO:1) is N-acetylated, and contains a C-7-amido-4-methylcoumarin (AMC) quenched fluorophore on its C-terminus. In the presence of LF, the quenched peptide substrate is cleaved at the N-terminus between the arginine (R) residue and the AMC quenched fluorophore derivative by the protease activity of the toxin, activating the fluorescence of the AMC fluorophore. If LF's protease activity is inhibited, the peptide substrate will not be cleaved, remaining in a quenched state. For these studies, LF (2 nM) was incubated with different amounts (5 μM-10 mM) of sulindac, fusaric acid and naproxen to monitor their ability to inhibit LF protease activity. Results showed sulindac to be the strongest inhibitor of LF proteases activity with an IC50 of 173 μM, while fusaric acid had a lower inhibitory effect with an IC50 of 530 μM (FIG. 6 a-b). Meanwhile, naproxen failed to inhibit LF, as expected since naproxen does not bind to the catalytic center of LF (FIG. 6 c). These results indicate that naproxen does not bind to the toxin's enzymatic pocket, while sulindac and fusaric acid bind and effectively inhibit LF's protease activity, as predicted by the computational docking studies.

vi. Inhibition of LeTx Cytotoxicity by Sulindac and Fusaric Acid

MAPPK cleavage by LF is the main reason for LF cytotoxicity in macrophages and inhibitors of LF protease activity could be used as potential inhibitors of the anthrax toxin cytotoxicity. To test whether sulindac or fusaric acid are inhibitors of the anthrax toxin, we treated RAW 264.7 macrophages (35,000 cells) with LeTx, a combination of PA (15 nM) and LF (5 nM), in the presence of various amounts of the corresponding inhibitor. Results show that sulindac exhibits a dose-dependent inhibition of LeTx with an IC50 of 31.7 μM (FIG. 6d). Fusaric acid also inhibited LeTx albeit with a higher IC50 (75.3 μM, FIG. 6e). As predicted by computational docking experiments and suggested by the lack of protease activity inhibition, naproxen failed to inhibit LeTx (FIG. 6f). These results show that sulindac, an FDA-approved anti-inflammatory drug, is the most potent inhibitor of the anthrax toxin as compared to the other two compounds tested. However, the FDA-approved anti-inflammatory drug naproxen did not prevent LeTx toxicity, presumably because this drug does not bind to the catalytic center of LF as the computational studies indicated. Although fusaric acid also inhibited LeTx toxicity, it is not as potent as sulindac and it is not FDA-approved, diminishing any interest to utilize this compound for the treatment of anthrax infection under a repurposing strategy.

vii. Sulindac Metabolic Derivatives as Inhibitors of LF Toxin

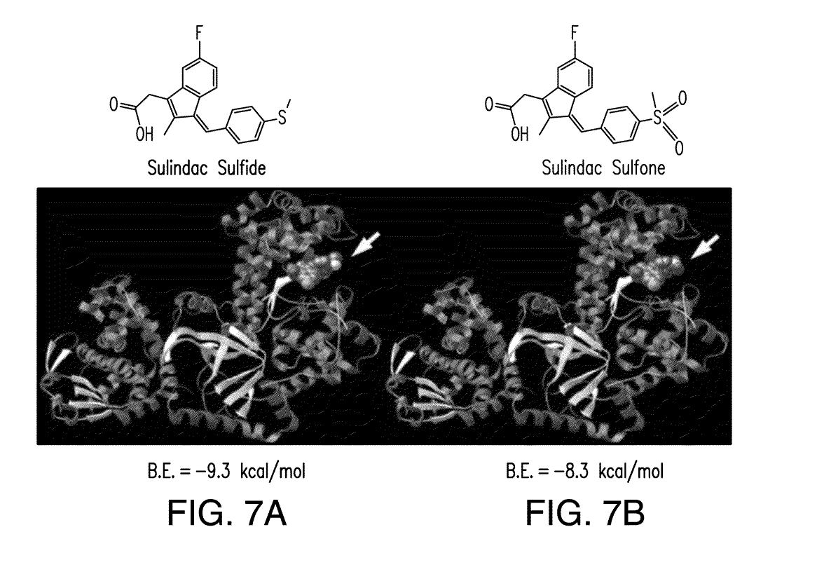

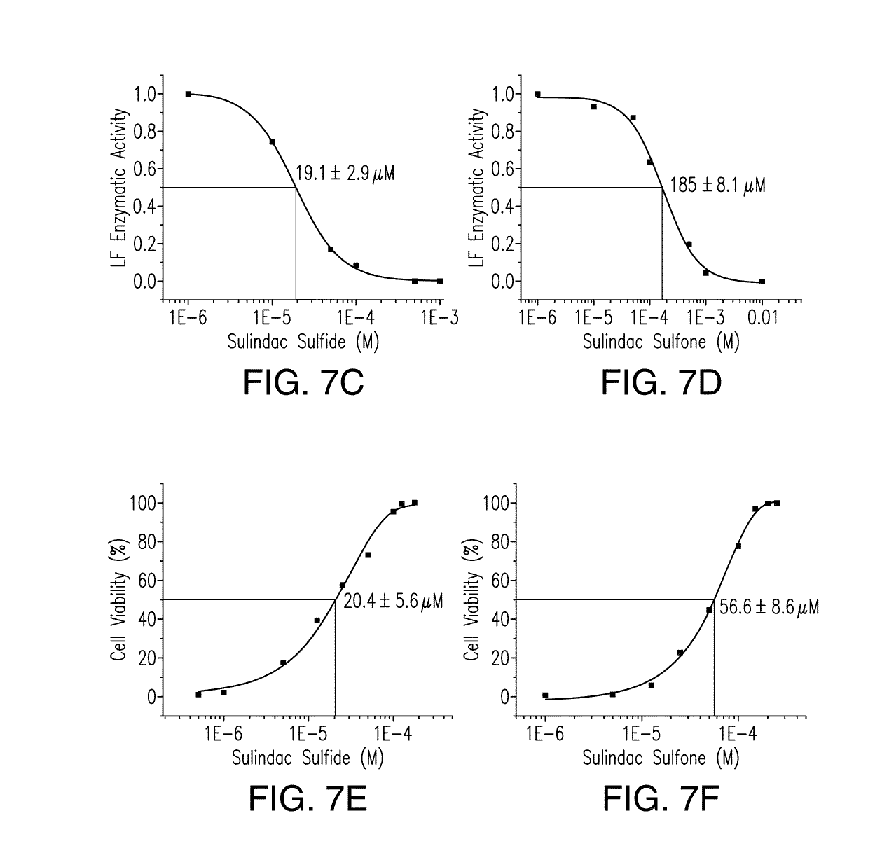

The toxicity, pharmacokinetic and metabolic profile of sulindac has been extensively studied in clinical trials. After a typical dosage of 150 mg, the maximum concentration of sulindac in human blood plasma is of 5.71±2.17 μg/mL, with a mean half-life of 7.8 h.28 Sulindac is actually a prodrug that upon oral administration is transformed by the liver to the reduced sulfide and the oxidized sulfone. Sulindac sulfide is the actual COX(I/II) inhibitor, while the sulindac sulfone has not been found to have an anti-inflammatory activity. Since sulindac is metabolized to the corresponding sulfone and sulfide, whether these metabolic products bind and inhibit LF similar to the parent drug was tested. First, computational docking studies were performed between the sulindac metabolites and LF. Studies with sulindac sulfide revealed similar results to those of sulindac with an even greater number of binding conformations at the catalytic center. After examining the most favorable binding conformations, it was observed that sulindac sulfide preferably bound to the catalytic site of LF with a binding energy of −9.3 kcal/mol (FIG. 7a). The predicted lower binding energy of the sulfide metabolite along with the multiple interactions at the catalytic site of LF indicate that this primary metabolite of sulindac can potentially be a stronger inhibitor of LF than sulindac itself. Furthermore, experiments with sulindac sulfone provided very similar results to those observed with both sulindac and sulindac sulfide. Most of the possible binding sites for sulindac sulfone in LF were also observed on the catalytic pocket and the hydrophobic domain III. The conformation of sulindac sulfone with the most favorable binding was observed at the catalytic pocket with a binding energy of −8.3 kcal/mol, a weaker interaction than that of sulindac and sulindac sulfide (FIG. 7b). Taken together, the docking studies indicate that sulindac sulfide and sulindac sulfone bind to the catalytic center of LF and potentially inhibit the toxin. Moreover, the higher bioavailability of sulindac sulfide after metabolic reduction of sulindac, makes this metabolite an ideal inhibitor of LF, since sulindac sulfide will be more readily available in circulation than sulindac sulfone.

LF protease activity inhibition studies also revealed that both sulindac sulfide and sulindac sulfone were good inhibitors of LF protease activity. For sulindac sulfide, an IC50 of 19.1 μM was observed (FIG. 7c), indicating that this metabolite could be a better inhibitor of the anthrax toxin that its parent drug sulindac (IC50=173 μM). In contrast, for sulindac sulfone an IC50 of 185 μM was observed (FIG. 7d). This value was higher than what was observed for sulindac sulfide, but close to the value obtained with the parent drug. Finally, LeTx cytotoxicity inhibition studies corroborated the LF protease activity inhibition studies showing that sulindac sulfide was a better inhibitor of the anthrax toxin cytotoxicity to RAW264.7 macrophages, with an IC50 of 20.4 μM (FIG. 7e). Sulindac sulfone had a higher IC50 value of 56.6 μM (FIG. 7f), while the parent drug displayed an IC50 value of 31.7 μM. Taken together, these results demonstrate that sulindac sulfide, the reduced metabolic product of sulindac, is a better inhibitor of the anthrax toxin as compared to the parent drug, sulindac, presumably because sulindac sulfide binds stronger to the LF catalytic center than sulindac or sulindac sulfone. Furthermore, these findings indicate that sulindac (Clinoril) can be used for the treatment for Anthrax, since both sulindac and its active metabolite sulindac sulfide strongly inhibit the Anthrax lethal factor.

viii. Sulindac bMR Nanosensors as Inhibition of LF.

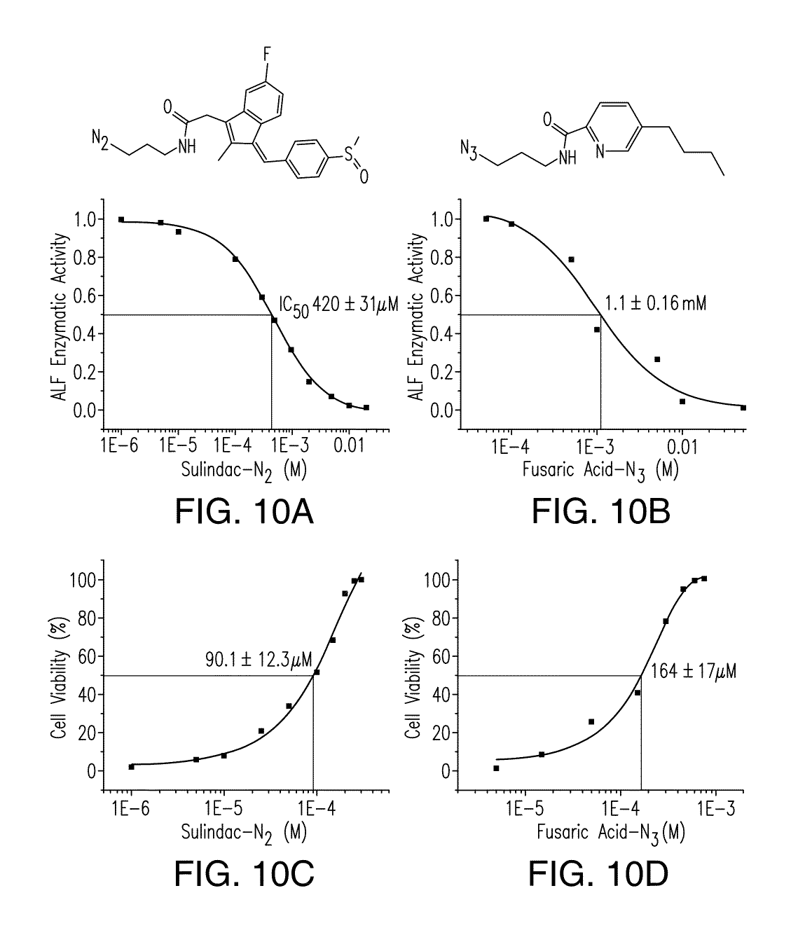

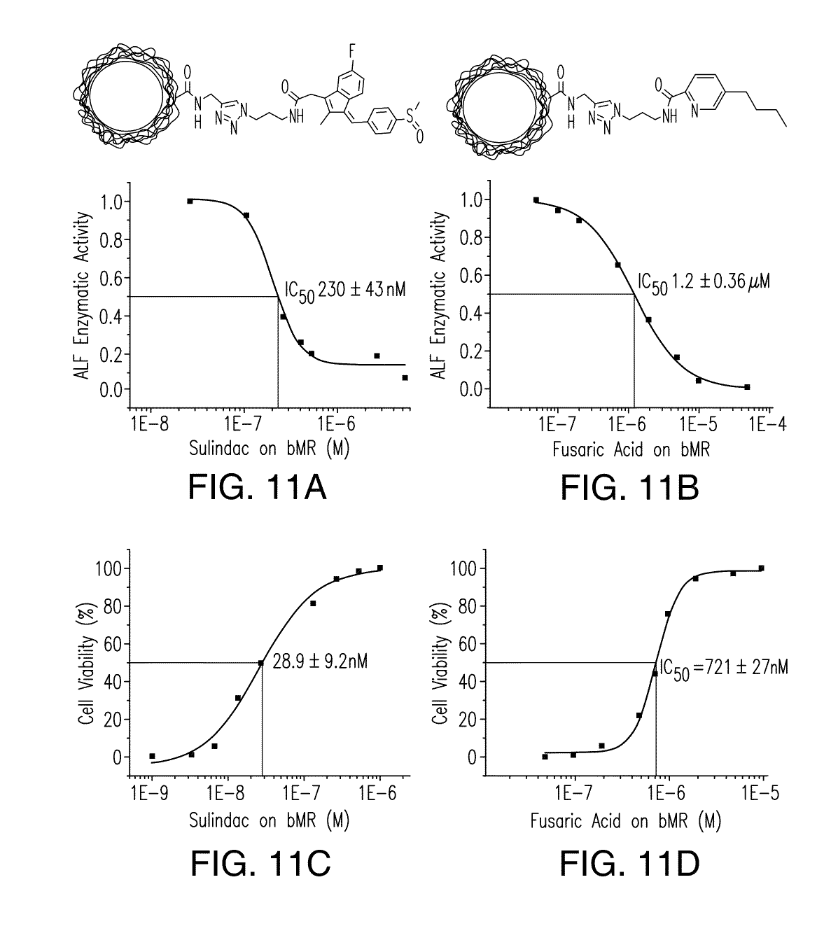

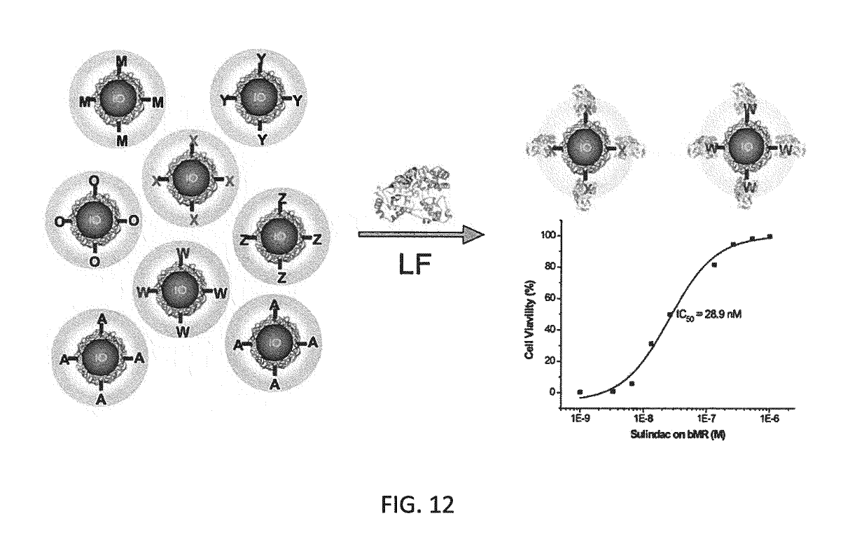

As the screenings were done using a nanosensor where sulindac is conjugated to the magnetic nanoparticles, it was reasoned that (1) sulindac carboxylic acid group is not involved in binding to LF since this functional group is utilized to conjugate the molecule to the nanoparticle and (2) the sulindac-bMR sensor itself can be used as a potential inhibitor of the toxin. To examine this hypothesis, if a sulindac molecule conjugated to an azide-containing linker (3-azidopropyl-1-amine) could still inhibit LF was investigated. This particular linker was selected, as it was the one utilized for the conjugation of sulindac to the magnetic nanoparticles in order to fabricate the sulindac bMR nanosensors of the screening studies. Results with the azide-linker conjugated molecule (sulindac-N3) showed that indeed this molecule inhibited LF by interfering with both the toxin's proteases activity (420 μM) and cytotoxicity to macrophages (90.1 μM) (FIGS. 10a and 10c). However, it is important to point out that modification of sulindac carboxylic acid with the azide linker reduced the inhibitory potency compared to the values obtained with sulindac. In contrast, when the sulindac-bMR nanosensor was tested, an IC50 of 0.23 μM (230 nM) for inhibition of LF proteases activity and 0.028 μM (28.9 nM) for inhibition of cytotoxicity to macrophages was obtained. These IC50 values were lower not only to those obtained using (sulindac-N3) but also to the IC50 values obtained with sulindac or its metabolic derivatives (FIG. 11a). A similar trend was observed when the fusaric acid-bMR was used to inhibit LF with an IC50 of 1.32 μM via the fluorogenic substrate assay (FIG. 11b) and 721 nM using RAW 264.7 macrophages (FIG. 11d). On the other hand, when fusaric acid-N3 was used the inhibition potency decreased, reporting an IC50 (fluorogenic substrate assay) of 1.1 mM and 164 μM (cell viability) (FIGS. 10b and 10d). The enhanced inhibitory properties of the sulindac-bMR and fusaric acid bMR nanosensors is not surprising, as it is well documented that the multiple display of low affinity ligands on polymers and nanoparticles results in a multivalent system with enhanced binding and inhibitory properties. Results are summarized in Table 5.

TABLE 5
Summary of the inhibitory concentrations of the modifications of sulindac and fusaric acid.

| | IC₅₀ (μM) | IC₅₀ (μM) |
|---|---|---|
| | Protease | Cell |
| Inhibitor | Activity | Viability |
| 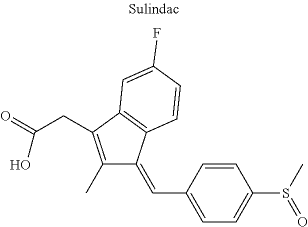 [SMILES: CC1=C(CC(=O)O)c2cc(F)ccc2/C1=C\c1ccc(S(C)=O)cc1] | 173 ± 8.7 | 31.7 ± 7.3 |
| 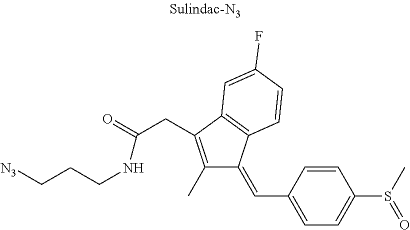 [SMILES: CC1=C(CC(=O)NCCCN=[N+]=[N-])c2cc(F)ccc2/C1=C\c1ccc(S(C)=O)cc1] | 420 ± 31 | 90.1 ± 12.3 |
| 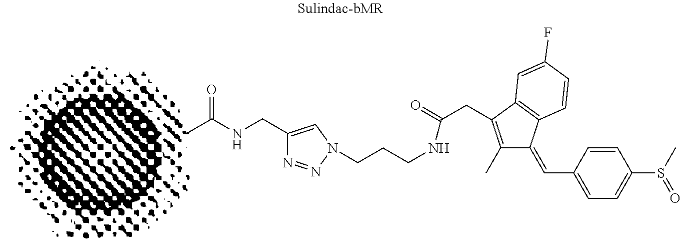 [SMILES: CC(=O)NCc1cn(CCCNC(=O)CC2=C(C)/C(=C/c3ccc(S(C)=O)cc3)c3ccc(F)cc32)nn1] | 0.23 ± 0.04 | 0.029 ± 0.01 |
| 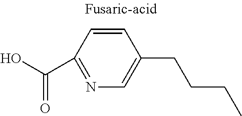 | 530 ± 41 | 75.3 ± 13.8 |
| 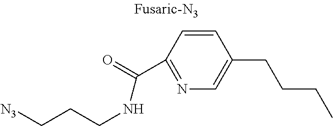 [SMILES: CCCCc1ccc(C(=O)NCCCN=[N+]=[N-])nc1] | 1100 ± 160 | 164 ± 17 |
| 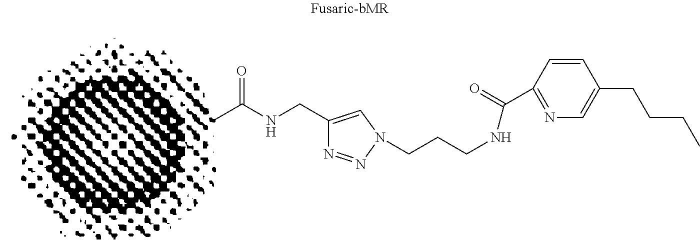 [SMILES: CCCCc1ccc(C(=O)NCCCn2cc(CNC(C)=O)nn2)nc1] | 1.2 ± 0.36 | 0.721 ± 0.03 |

Disclosed is a method to identify small molecules as toxin inhibitors using bMR nanosensors. Upon screening a library of small molecules via magnetic relaxation, three small molecules that bind to the anthrax lethal factor (LF) were identified. Further biological screening identified sulindac and its metabolic byproducts as micromolar inhibitors of LF toxicity. Sulindac is an FDA-approved drug. Furthermore, both sulindac- and fusaric acid-bMR nanosensors were evaluated as potential multivalent therapeutics finding nanomolar inhibition for both LF protease activity and macrophage toxicity. The method is unique as it performs the binding screening on a magnetic nanoparticle platform that subsequently can be used for inhibitory screening studies. In addition, the method is done in solution and can be easily adapted to screen other protein-drug interactions. Taken together, using the bMR nanosensor screening method, sulindac by itself and sulindac conjugated on the surface of a nanoparticle (sulindac bMR) have been identified as potent inhibitors of the anthrax lethal factor toxin. Used in combination with antibiotics, these drugs and drug-bMR conjugates can be used to inhibit the toxins that remain in circulation after the bacterial levels have been reduced by the antibiotic.

### REFERENCES

1. Kaittanis, C.; Nath, S.; Perez, J. M., Rapid nanoparticle-mediated monitoring of bacterial metabolic activity and assessment of antimicrobial susceptibility in blood with magnetic relaxation. PLoS One 2008, 3 (9), e3253.
2. Kaittanis, C.; Naser, S. A.; Perez, J. M., One-step, nanoparticle-mediated bacterial detection with magnetic relaxation. Nano Lett 2007, 7 (2), 380-3.
3. Kaittanis, C.; Santra, S.; Perez, J. M., Role of nanoparticle valency in the nondestructive magnetic-relaxation-mediated detection and magnetic isolation of cells in complex media. J Am Chem Soc 2009, 131 (35), 12780-91.
4. Perez, J. M.; Grimm, J.; Josephson, L.; Weissleder, R., Integrated nanosensors to determine levels and functional activity of human telomerase. Neoplasia 2008, 10 (10), 1066-72.
5. Haun, J. B.; Castro, C. M.; Wang, R.; Peterson, V. M.; Marinelli, B. S.; Lee, H.; Weissleder, R., Micro-NMR for rapid molecular analysis of human tumor samples. Sci Transl Med 2011, 3 (71), 71ra16.
6. Santiesteban, O. J.; Kaittanis, C.; Perez, J. M., Assessment of Molecular Interactions through Magnetic Relaxation. Angew Chem Int Ed Engl 2012, 51 (27), 6728-32.
7. Jernigan, J. A.; Stephens, D. S.; Ashford, D. A.; Omenaca, C.; Topiel, M. S.; Galbraith, M.; Tapper, M.; Fisk, T. L.; Zaki, S.; Popovic, T.; Meyer, R. F.; Quinn, C. P.; Harper, S. A.; Fridkin, S. K.; Sejvar, J. J.; Shepard, C. W.; McConnell, M.; Guarner, J.; Shieh, W. J.; Malecki, J. M.; Gerberding, J. L.; Hughes, J. M.; Perkins, B. A., Bioterrorism-related inhalational anthrax: the first 10 cases reported in the United States. Emerg Infect Dis 2001, 7 (6), 933-44.
8. Mock, M.; Mignot, T., Anthrax toxins and the host: a story of intimacy. Cell Microbiol 2003, 5 (1), 15-23.
9. Collier, R. J., Membrane translocation by anthrax toxin. Mol Aspects Med 2009, 30 (6), 413-22.
10. Collier, R. J., Mechanism of membrane translocation by anthrax toxin: insertion and pore formation by protective antigen. J Appl Microbiol 1999, 87 (2), 283.
11. Bann, J. G, Anthrax toxin protective antigen—insights into molecular switching from prepore to pore. Protein Sci 2011, 21 (1), 1-12.
12. Moayeri, M.; Leppla, S. H., Cellular and systemic effects of anthrax lethal toxin and edema toxin. Mol Aspects Med 2009, 30 (6), 439-55.
13. Alileche, A.; Serfass, E. R.; Muehlbauer, S. M.; Porcelli, S. A.; Brojatsch, J., Anthrax lethal toxin-mediated killing of human and murine dendritic cells impairs the adaptive immune response. PLoS Pathog 2005, 1 (2), e19.
14. Vitale, G.; Bernardi, L.; Napolitani, G; Mock, M.; Montecucco, C., Susceptibility of mitogen-activated protein kinase kinase family members to proteolysis by anthrax lethal factor. Biochem J 2000, 352 Pt 3, 739-45.
15. Park, H. C.; Sung, S. R.; Lim, S. M.; Lee, J. S.; Kim, S. K.; Yoon, M. Y, Proteolytic assay-based screening identifies a potent inhibitor of anthrax lethal factor. Microb Pathog 2012, 53 (2), 109-12.
16. Panchal, R. G.; Hermone, A. R.; Nguyen, T. L.; Wong, T. Y; Schwarzenbacher, R.; Schmidt, J.; Lane, D.; McGrath, C.; Turk, B. E.; Burnett, J.; Aman, M. J.; Little, S.; Sausville, E. A.; Zaharevitz, D. W.; Cantley, L. C.; Liddington, R. C.; Gussio, R.; Bavari, S., Identification of small molecule inhibitors of anthrax lethal factor. Nat Struct Mol Biol 2004, 11 (1), 67-72.
17. Forino, M.; Johnson, S.; Wong, T. Y; Rozanov, D. V.; Savinov, A. Y; Li, W.; Fattorusso, R.; Becattini, B.; Orry, A. J.; Jung, D.; Abagyan, R. A.; Smith, J. W.; Alibek, K.; Liddington, R. C.; Strongin, A. Y; Pellecchia, M., Efficient synthetic inhibitors of anthrax lethal factor. Proc Natl Acad Sci USA 2005, 102 (27), 9499-504.
18. Johnson, S. L.; Chen, L. H.; Barile, E.; Emdadi, A.; Sabet, M.; Yuan, H.; Wei, J.; Guiney, D.; Pellecchia, M., Structure-activity relationship studies of a novel series of anthrax lethal factor inhibitors. Bioorg Med Chem 2009, 17 (9), 3352-68.
19. Bannwarth, L.; Goldberg, A. B.; Chen, C.; Turk, B. E., Identification of exosite-targeting inhibitors of anthrax lethal factor by high-throughput screening. Chem Biol 2012, 19 (7), 875-82.
20. Wei, D.; Bu, Z.; Yu, A.; Li, F., Identification of a lead small-molecule inhibitor of anthrax lethal toxin by using fluorescence-based high-throughput screening. BMB Rep 2011, 44 (12), 811-5.
21. Santra, S.; Kaittanis, C.; Grimm, J.; Perez, J. M., Drug/dye-loaded, multifunctional iron oxide nanoparticles for combined targeted cancer therapy and dual optical/magnetic resonance imaging. Small 2009, 5 (16), 1862-8.
22. Cryer, B.; Feldman, M., Cyclooxygenase-1 and cyclooxygenase-2 selectivity of widely used nonsteroidal anti-inflammatory drugs. Am J Med 1998, 104 (5), 413-21.
23. Wang, H.; Ng, T. B., Pharmacological activities of fusaric acid (5-butylpicolinic acid). Life Sci 1999, 65 (9), 849-56.
24. Pannifer, A. D.; Wong, T. Y.; Schwarzenbacher, R.; Renatus, M.; Petosa, C.; Bienkowska, J.; Lacy, D. B.; Collier, R. J.; Park, S.; Leppla, S. H.; Hanna, P.; Liddington, R. C., Crystal structure of the anthrax lethal factor. Nature 2001, 414 (6860), 229-33.
25. Morris, G M.; Goodsell, D. S.; Halliday, R. S.; Huey, R.; Hart, W. E.; Belew, R. K.; Olson, A. J., Automated docking using a Lamarckian genetic algorithm and an empirical binding free energy function. Journal of Computational Chemistry 1998, 19 (14), 1639-1662.
26. Morris, G. M.; Huey, R.; Lindstrom, W.; Sanner, M. F.; Belew, R. K.; Goodsell, D. S.; Olson, A. J., AutoDock4 and AutoDockTools4: Automated docking with selective receptor flexibility. J Comput Chem 2009, 30 (16), 2785-91.
27. Tonello, F.; Seveso, M.; Marin, O.; Mock, M.; Montecucco, C., Screening inhibitors of anthrax lethal factor. Nature 2002, 418 (6896), 386.
28. Reid, J. M.; Mandrekar, S. J.; Carlson, E. C.; Harmsen, W. S.; Green, E. M.; McGovern, R. M.; Szabo, E.; Ames, M. M.; Boring, D.; Limburg, P. J., Comparative bioavailability of sulindac in capsule and tablet compositions. Cancer Epidemiol Biomarkers Prev 2008, 17 (3), 674-9.
29. Mattila, J.; Mantyla, R.; Vuorela, A.; Lamminsivu, U.; Mannisto, P., Pharmacokinetics of graded oral doses of sulindac in man. Arzneimittelforschung 1984, 34 (2), 226-9.
30. Reddy, B. S.; Kawamori, T.; Lubet, R. A.; Steele, V. E.; Kelloff, G. J.; Rao, C. V., Chemopreventive efficacy of sulindac sulfone against colon cancer depends on time of administration during carcinogenic process. Cancer Res 1999, 59 (14), 3387-91.
31. Duggan, D. E.; Hooke, K. F.; Risley, E. A.; Shen, T. Y.; Arman, C. G, Identification of the biologically active form of sulindac. J Pharmacol Exp Ther 1977, 201 (1), 8-13.
32. Piazza, G A.; Alberts, D. S.; Hixson, L. J.; Paranka, N. S.; Li, H.; Finn, T.; Bogert, C.; Guillen, J. M.; Brendel, K.; Gross, P. H.; Sperl, G.; Ritchie, J.; Burt, R. W.; Ellsworth, L.; Ahnen, D. J.; Pamukcu, R., Sulindac sulfone inhibits azoxymethane-induced colon carcinogenesis in rats without reducing prostaglandin levels. Cancer Res 1997, 57 (14), 2909-15.
33. Kaittanis, C.; Santra, S.; Santiesteban, O. J.; Henderson, T. J.; Perez, J. M., The assembly state between magnetic nanosensors and their targets orchestrates their magnetic relaxation response. J Am Chem Soc 2011, 133 (10), 3668-76.
34. Bowman, M. C.; Ballard, T. E.; Ackerson, C. J.; Feldheim, D. L.; Margolis, D. M.; Melander, C., Inhibition of HIV fusion with multivalent gold nanoparticles. J Am Chem Soc 2008, 130 (22), 6896-7.
35. Carboni, B.; Benalil, A.; Vaultier, M., Aliphatic amino azides as key building blocks for efficient polyamine syntheses. The Journal of Organic Chemistry 1993, 58 (14), 3736-3741.

What is claimed is:

1. A method of inhibiting anthrax lethal factor (LF) toxin activity comprising administering an effective amount of one or more carboxylic acid-containing small molecules to a subject in need thereof.

2. The method of claim 1, wherein a carboxylic acid-containing small molecule is Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, or a derivative thereof, or mixtures thereof.

3. The method of claim 1, wherein the carboxylic acid-containing small molecule binds to LF present in the subject.

4. The method of claim 3, wherein the binding occurs at the active site of LF.

5. The method of claim 3, wherein the one or more carboxylic acid-containing small molecules allosterically inhibit the LF toxin activity.

6. The method of claim 1, wherein the subject in need thereof is a subject infected with anthrax.

7. The method of claim 1, wherein the one or more carboxylic acid-containing small molecule is conjugated to a nanoparticle.

8. A method of treating a subject having anthrax comprising administering a therapeutically effective amount of a composition comprising one or more carboxylic acid-containing small molecules to the subject, wherein the effective amount of the composition comprising a carboxylic acid-containing small molecule reduces or inhibits lethal factor protease activity.

9. The method of claim 8, wherein a carboxylic acid-containing small molecule is Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, a derivative thereof, or a mixture thereof.

10. The method of claim 9, further comprising administering a composition comprising an anthrax therapeutic agent.

11. The method of claim 8, wherein the subject in need thereof is a subject infected with anthrax.

12. A method of screening comprising
a) conjugating a magnetic relaxing (MR) nanosensor to a ligand to form a ligand-MR nanosensor complex;
b) contacting the ligand-MR nanosensor complex with a sample containing possible ligand targets; and
c) measuring the magnetic resonance in the sample, wherein a change in magnetic resonance is indicative of a target bound to the ligand-MR nanosensor complex.

13. The method of claim 12, wherein the ligand is a known compound.

14. The method of claim 12, wherein the ligand is a carboxylic acid-containing molecule.

15. The method of claim 13, wherein a carboxylic acid-containing molecule is Sulindac, Bezafibrate, Ketoprofen, Indometacin, Ibuprofen, Retinoic Acid, (S)-(+)-6-Methoxy-α-methyl-2-naphthaleneacetic acid, Homovanillic Acid, (±)-α-Lipoic acid, Nalidixic Acid, L-Mimosine, N-Hippuryl-His-Leu Hydrate, Acemetacin, Mefenamic Acid, Cetirizine dihydrochloride, Furosemide, Rebamipide Hydrate, Bumetanide, Aristolochic Acid I, Etodolac, Fusaric Acid, R(+)-IAA-94, Tamibarotene, NS3694, Sivelestat sodium salt hydrate, Oxaprozin, GW9508, Raltitrexed monohydrate, Rhein, and Doxorubicin, derivatives thereof, or mixtures thereof.

16. The method of claim 12, wherein the MR nanosensor comprises an iron oxide nanoparticle.

17. The method of claim 13, wherein the ligand is sulindac.

18. The method of claim 12, wherein the target is anthrax toxin.

19. The method of claim 18, wherein the anthrax toxin is LF.

20. The method of claim 12, wherein the change in magnetic resonance is an increase in magnetic resonance.
